SEQUENCE LISTING (69 sequences)

SEQ ID NO: 1 - DNA, 24 bp, Artificial Sequence
  Artificial Sequence source is human.
tataaacttgtggtagttggacct

SEQ ID NO: 2 - DNA, 20 bp, Artificial Sequence
  Artificial Sequence source is human.
cgtccacaaaatgattctga

SEQ ID NO: 3 - DNA, 21 bp, Artificial Sequence
  Artificial Sequence source is human.
tcagcaaagacaagacaggta

SEQ ID NO: 4 - DNA, 18 bp, Artificial Sequence
  Artificial Sequence source is human.
agcaatgccctctcaaga

SEQ ID NO: 5 - DNA, 24 bp, Artificial Sequence
  Artificial Sequence source is human.
agtaaaaggtgcactgtaataatc

SEQ ID NO: 6 - DNA, 23 bp, Artificial Sequence
  Artificial Sequence source is human.
gtgtcgagaatatccaagagcca

SEQ ID NO: 7 - DNA, 28 bp, Artificial Sequence
  Artificial Sequence source is human.
tatcgtcaaggcactcttgcctacgcca

SEQ ID NO: 8 - DNA, 28 bp, Artificial Sequence
  Artificial Sequence source is human.
gactgtgtttctcccttctcaggattcc

SEQ ID NO: 9 - DNA, 34 bp, Artificial Sequence
  Artificial Sequence source is human.
gacataacagttatgattttgcagaaaacagatc

SEQ ID NO: 10 - DNA, 23 bp, Artificial Sequence
  Artificial Sequence source is human.
ggcctgctgaaaatgactgaata

SEQ ID NO: 11 - DNA, 23 bp, Artificial Sequence
  Artificial Sequence source is human.
ctcatgaaaatggtcagagaaac

SEQ ID NO: 12 - DNA, 27 bp, Artificial Sequence
  Artificial Sequence source is human.
gaatataaacttgtggtagttggacct

SEQ ID NO: 13 - DNA, 21 bp, Artificial Sequence
  Artificial Sequence source is human.
atcaaagaatggtcctgcacc

SEQ ID NO: 14 - DNA, 40 bp, Artificial Sequence
  Artificial Sequence source is human.
gcgagctatcgtcaaggcactcttgcctacgccagctcgc

SEQ ID NO: 15 - DNA, 44 bp, Artificial Sequence
  Artificial Sequence source is human.
ccgagcgacataacagttatgattttgcagaaaacagagctcgg

SEQ ID NO: 16 - DNA, 40 bp, Artificial Sequence
  Artificial Sequence source is human.
gcgagcaagccttcgcctgtcctcatgtattggtgctcgc

SEQ ID NO: 17 - DNA, 40 bp, Artificial Sequence
  Artificial Sequence source is human.
gcgagcgactgtgtttctcccttctcaggattccgctcgc

SEQ ID NO: 18 - DNA, 23 bp, Artificial Sequence
  Artificial Sequence source is human.
ggatattctcgacacagcaggtt

SEQ ID NO: 19 - DNA, 46 bp, Artificial Sequence
  Artificial Sequence source is human.
gcgagcgacataacagttatgattttgcagaaaacagatcgctcgc

SEQ ID NO: 20 - DNA, 41 bp, Artificial Sequence
  Artificial Sequence source is human.
gcgagcctatcgtcaaggcactcttgcctacgccagctcgc

SEQ ID NO: 21 - DNA, 41 bp, Artificial Sequence
  Artificial Sequence source is human.
gcgagcaggaatcctgagaagggagaaacacagtcgctcgc

SEQ ID NO: 22 - DNA, 24 bp, Artificial Sequence
  Artificial Sequence source is human.
gtagtaattgatggagaaacctgt

SEQ ID NO: 23 - DNA, 25 bp, Artificial Sequence
  Artificial Sequence source is human.
tggacatactggatacagctggact

SEQ ID NO: 24 - DNA, 17 bp, Artificial Sequence
  Artificial Sequence source is human.
cggcccctcgcgcttta

SEQ ID NO: 25 - DNA, 23 bp, Artificial Sequence
  Artificial Sequence source is human.
agctgtgtcggcccaggactgca

SEQ ID NO: 26 - DNA, 21 bp, Artificial Sequence
  Artificial Sequence source is human.
atgtgacccagcggcccctcg

SEQ ID NO: 27 - DNA, 25 bp, Artificial Sequence
  Artificial Sequence source is human.
ctataatggtgaatatcttcaaatg

SEQ ID NO: 28 - DNA, 24 bp, Artificial Sequence
  Artificial Sequence source is human.
agtacaaactggtggtgcctggag

SEQ ID NO: 29 - DNA, 22 bp, Artificial Sequence
  Artificial Sequence source is human.
actggtggtggttccagcaggt

SEQ ID NO: 30 - DNA, 28 bp, Artificial Sequence
  Artificial Sequence source is human.
atataaacttgtggtagttccagctggt

SEQ ID NO: 31 - DNA, 21 bp, Artificial Sequence
  Artificial Sequence source is human.
ggttctggattagctggattg

SEQ ID NO: 32 - DNA, 22 bp, Artificial Sequence
  Artificial Sequence source is human.
ggatattctcgacacagcaggc

SEQ ID NO: 33 - DNA, 20 bp, Artificial Sequence
  Artificial Sequence source is human.
gggagacgtgcctgttggac

SEQ ID NO: 34 - DNA, 22 bp, Artificial Sequence
  Artificial Sequence source is human.
ttgatggcaaacacacacagga

SEQ ID NO: 35 - DNA, 24 bp, Artificial Sequence
  Artificial Sequence source is human.
acaagtggttatagatggtgaaac

SEQ ID NO: 36 - DNA, 21 bp, Artificial Sequence
  Artificial Sequence source is human.
tgatggcaaatacacagagga

SEQ ID NO: 37 - DNA, 22 bp, Artificial Sequence
  Artificial Sequence source is human.
ggacatactggatacagctggc

SEQ ID NO: 38 - DNA, 23 bp, Artificial Sequence
  Artificial Sequence source is human.
ttggacatcctggataccgctgg

SEQ ID NO: 39 - DNA, 21 bp, Artificial Sequence
  Artificial Sequence source is human.
ccctgaggagcgatgacggaa

SEQ ID NO: 40 - DNA, 19 bp, Artificial Sequence
  Artificial Sequence source is human.
agtggggtcgtattcgtcc

SEQ ID NO: 41 - DNA, 22 bp, Artificial Sequence
  Artificial Sequence source is human.
tcacctctatagtggggtcgta

SEQ ID NO: 42 - DNA, 22 bp, Artificial Sequence
  Artificial Sequence source is human.
gttcttgctggtgtgaaatgac

SEQ ID NO: 43 - DNA, 26 bp, Artificial Sequence
  Artificial Sequence source is human.
aggtccttgctggtgtgaaatgactg

SEQ ID NO: 44 - DNA, 27 bp, Artificial Sequence
  Artificial Sequence source is human.
gtggttctggattagctggattgtcag

SEQ ID NO: 45 - DNA, 25 bp, Artificial Sequence
  Artificial Sequence source is human.
gttggacatactggatacagctggc

SEQ ID NO: 46 - DNA, 25 bp, Artificial Sequence
  Artificial Sequence source is human.
ggcaaatacacagaggaagccttcg

SEQ ID NO: 47 - DNA, 27 bp, Artificial Sequence
  Artificial Sequence source is human.
gttggacatactggatacagctggact

SEQ ID NO: 48 - DNA, 24 bp, Artificial Sequence
  Artificial Sequence source is human.
aatataagctggtggtgccgggcg

SEQ ID NO: 49 - DNA, 22 bp, Artificial Sequence
  Artificial Sequence source is human.
ataagctggtggtgcccgccgt

SEQ ID NO: 50 - DNA, 28 bp, Artificial Sequence
  Artificial Sequence source is human.
tgaatataaacttgtggtacctggagct

SEQ ID NO: 51 - DNA, 25 bp, Artificial Sequence
  Artificial Sequence source is human.
aatataagctggtggtgccgggcgc

SEQ ID NO: 52 - DNA, 23 bp, Artificial Sequence
  Artificial Sequence source is human.
aatggttctggatcagctggatg

SEQ ID NO: 53 - DNA, 24 bp, Artificial Sequence
  Artificial Sequence source is human.
ataagctggtggtggtgcccgccg

SEQ ID NO: 54 - DNA, 26 bp, Artificial Sequence
  Artificial Sequence source is human.
tatagatggtgaaacctgtttgttgg

SEQ ID NO: 55 - DNA, 24 bp, Artificial Sequence
  Artificial Sequence source is human.
ctattattgatggcaaccacacag

SEQ ID NO: 56 - DNA, 32 bp, Artificial Sequence
  Artificial Sequence source is human.
gcgagcgtggtgttgggaaaagcgcagctcgc

SEQ ID NO: 57 - DNA, 35 bp, Artificial Sequence
  Artificial Sequence source is human.
gcgagccgtcggtgtgggcagagtgcgctgctcgc

SEQ ID NO: 58 - DNA, 36 bp, Artificial Sequence
  Artificial Sequence source is human.
gcgagcgaaacctcagccaagaccagacaggctcgc

SEQ ID NO: 59 - DNA, 46 bp, Artificial Sequence; identical to SEQ ID NO: 19
  Artificial Sequence source is human.

SEQ ID NO: 60 - DNA, 25 bp, Artificial Sequence
  Artificial Sequence source is human.
atataaacttgtggtacctggagct

SEQ ID NO: 61 - DNA, 22 bp, Artificial Sequence
  Artificial Sequence source is human.
tatagatggtgaaacctgtttg

SEQ ID NO: 62 - DNA, 29 bp, Artificial Sequence
  Artificial Sequence source is human.
cttgctattattgatggcaaccacacaga

SEQ ID NO: 63 - DNA, 22 bp, Artificial Sequence
  Artificial Sequence source is human.
tatagatggtgaaacctgtttg

SEQ ID NO: 64 - DNA, 21 bp, Artificial Sequence
  Artificial Sequence source is human.
ataagctggtggtgccgggcg

SEQ ID NO: 65 - DNA, 19 bp, Artificial Sequence
  Artificial Sequence source is human.
atggttctggatcagctgg

SEQ ID NO: 66 - DNA, 24 bp, Artificial Sequence
  Artificial Sequence source is human.
aatataagctggtggtgccgggcg

SEQ ID NO: 67 - DNA, 26 bp, Artificial Sequence
  Artificial Sequence source is human.
aatggttctggatcagctggatggtc

SEQ ID NO: 68 - DNA, 33 bp, Artificial Sequence
  Artificial Sequence source is human.
gcgagcgtggtgttggggaaaagcgcagctcgc

SEQ ID NO: 69 - DNA, 36 bp, Artificial Sequence
  Artificial Sequence source is human.
gcgagccgtcggtgtgggcaagagtgcgctgctcgc

(19) United States
(12) Patent Application Publication
(10) Pub. No.: US 2004/0106109 A1
(43) Pub. Date: Jun. 3, 2004

(54) Detection of ras mutations

(76) Inventors: Robert T Belly, Webster, NY (US); Alison V Todd, Glebe (AU); Caroline J Fuery, Toongabie (AU)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/110,707

(22) PCT Filed: Oct. 2, 2001
(86) PCT No.: PCT/US01/42422

(51) Int. Cl. C12Q 1/68; C07H 21/04

(52) U.S. Cl. 435/6; 536/24.3

(57) ABSTRACT

Mutations in K-ras, N-ras, and H-ras were determined using target specific primers and probes in REMS-PCR methods, nested PCR methods employing a restriction endonuclease, and REMS-PCR methods using molecular beacons.

### FIELD OF THE INVENTION

The present invention relates to primers, probes and methods for determining the presence of mutations. More specifically it relates to primers, probes and molecular beacons for determining the presence of ras mutations using Restriction Mediated Selection Polymerase Chain Reaction (REMS-PCR) and nested PCR methods.

### BACKGROUND OF THE INVENTION

The ras family of oncogenes (K-ras, H-ras, and N-ras) encode for membrane proteins possessing GTPase activity. These proteins are involved in cellular signal transduction. Specific point mutations, usually within the ras codons 12, 13, or 61, can result in the activation of these protooncogenes and result in subsequent neoplasia (Bos, J. L., 1989, Can. Res. 49:4682-4689).

The frequency with which ras mutations occur varies among different tumor types. The highest frequency is found in tumors from the exocrine pancreas, where more than 80% of tumors harbor a mutated K-ras gene (Bos et al., 1989, Can. Res. 49:4682-4689). H-ras mutations occur more frequently than N-ras and K-ras mutations in urinary tract tumors. The frequency of H-ras oncogene mutation has been estimated at 5% to 17% (Saito, S. et al., 1996, Int. J. Urol. 4:178-185). H-ras oncogene mutations have been detected in the urine of patients with bladder tumors (Haliassos, A. et al., 1992, Int. J. Oncol. 1:731-734); potentially representing a non-invasive means for detecting neoplasia. H-ras mutations have been reported in other cancers including thyroid and kidney carcinomas (Bos, J. L., 1988, Mutat. Res. 195:255-71), and human primary breast carcinomas (Theillet, C. et al., 1986, Cancer Res. 46:4776-4781).

Mutations of the N-ras gene are most commonly found in myeloid and lymphoid cancers. Bos (1988, Mutat. Res. 195:255-71) reported that about one-third of leukemia patients have a mutated ras gene, mostly N-ras, in both early stage pre-leukemia and acute myeloid leukemia. N-ras mutations have also been reported in human lymphoid malignancies (Neri, A. et al., 1998, Proc. Natl. Acad. Sci., USA, 85:9268-9272). More rarely, N-ras mutations have been detected in other carcinomas including melanoma; and carcinomas of the liver and thyroid.

Approximately 40-50% of colon cancers exhibit a mutation in the c-K-ras gene, with 86% of these mutations occurring at codons 12 and 13 (Bos, J. L. et al., 1987, Nature 327: (6120)293-7, Vogelstein B. et al., 1988, N. Engl. J. Med. 319:525-532). Ras mutations result in increased cell proliferation due to decreased intrinsic GTP-ase activity of the ras protein.

Lymph node metastasis is an important predictor of prognosis in colorectal carcinoma (Calaluce R et al., 1998, J. Surg. Oncol. 67:194-202). Turnbull et al. (1967, Ann. Surg. 166:420-7) extended the original classification of adenocarcinoma of the colon (Dukes, C. E. 1932, J. Pathol. Bacteriol. 35:323-332) into four clinicopathologic stages: Stage A-Tumor confined to the colon and its coats; Stage B-Tumor extension into pericolic fat; Stage C-Tumor metastasis to regional mesenteric lymph nodes, but no evidence of distant spread; Stage D-Tumor metastasis to liver, lung, bone. Although adjuvant therapies are of considerable benefit in Dukes C (stage III) colon cancer, no statistical benefit of adjuvant treatment has been demonstrated in Dukes B patients (Moertel C. G. et al., 1990, N. Engl. J. Med. 322:352-8). Thus, Dukes B patients generally do not receive adjuvant therapy after surgery. Approximately 20-30% of these patients will develop metastatic disease.

In a large multicenter study of 2721 patients including a total of 1173 Dukes' B, multivariate analysis suggested that the presence of a ras mutation increased risk of recurrence and death in all Dukes' stages (Andreyev, H. J. N. et al., 1998, Natl. Cancer Inst. 90 (9):675-684). Risk of recurrence and death increased with higher Dukes' stage. A study from the Southwest Oncology Group, concluded that mutation of the Ki-ras gene occurred in 41% of colon cancers and was associated with poor prognosis in stage II, but not stage III. In stage II, the 7-year survival rate of patients having a ras muation was 58%; whereas, the 7-year survival rate of patients with wild type ras was 86% (Ahnen, D. J. et al., 1998, Can. Res. 58:1149-1158).

Recently workers have examined the utility of determining mutations in K-ras as a means for sensitive detection of lymph node metastases in colorectal cancer. Hayashi et al. (1994, Cancer Res. 54:3853-3856) used a mutant allele-specific amplification (MASA) method to examine the lymph nodes of 22 colorectal cancer patients who were positive for either a K-ras or p53 mutation in the primary tumor. Seven of 14 cases in which genetic alterations were detected in lymph nodes had negative lymph nodes as determined by histology. In a subsequent study (Haysahi, N. et al., 1995, Lancet 345: 1257-1259), 120 colorectal cancer patients who had negative lymph nodes by histology were screened. Of 37 patients with genetically positive lymph nodes, 27 had a tumor recurrence within 5 years of surgery; whereas, none of the 34 patients with nodes that were negative by the molecular assay had a recurrence. Nakamori et al. (1997, Dis. Colon Rectum: 40 (Suppl 10):S29-36) have reported that either K-ras or p53 mutations were detected in 9 lymph nodes from a total of 17 patients who had these mutations in the primary tumor. Two of the nine patients with mutation-positive lymph nodes presented with recurrences to the liver, and all eight patients with mutation-negative lymph nodes remained disease-free. It has been reported (Monserrat S. et al., 1999, Clin. Cancer. Res. 5:2450-2454) that K-ras and p53 gene mutations as well as P16 promoter hypermethylations can be used to screen for lymph node metastasis in colorectal cancer patients, that molecular-based methods increase the sensitivity of tumor cell detection, and are a good predictor of recurrence in patients with resectable liver metastasis.

As pointed out by Andreyev et al. (1998, Natl. Cancer Inst. 90 (9):675-684), more than 75 research groups have published data on the significance of a K-ras gene mutation in colorectal cancer. Some workers have suggested that the presence of a K-ras mutation conveys prognostic significance, and other workers have reached the opposite conclusion. The discrepancy, in part, can be explained by the variable sensitivity of methods used for detecting ras mutations and the difficulty in determining mutations in the presence of excess wild type sequences. Furthermore, contamination during sample preparation and PCR amplification can be a serious problem.

Thus, a need exists for determining ras mutations, and other nucleic acid mutations particularly in the presence of high levels of wild type nucleic acid, using methods that are rapid, sensitive, specific and are capable of being automated. It is desirable to have available methods that reduce contamination.

### SUMMARY OF THE INVENTION

In accordance with the above-mentioned needs the present invention provides methods for amplifying and determining one or more mutations in one or more nucleic acids.

The present invention provides nucleic acid primers and probes for amplifying and determining ras mutations.

The invention provides REMS-PCR methods for determining ras mutations.

The invention provides REMS-PCR methods for determining ras mutations in the presence of excess wild type nucleic acid.

The invention provides primers, probes and nested PCR methods using one or more restriction endonucleases for amplifying and determining ras mutations.

The invention provides primers, probes and nested PCR methods using one or more restriction endonucleases for amplifying and determining ras mutations in the presence of excess wild type nucleic acid.

The invention provides primers, probes and nested PCR methods using one or more restriction endonucleases for amplifying and determining ras mutations in samples having a low copy number of the target nucleic acid.

In one embodiment the invention is practiced using means such as containment devices for reducing contamination and methods that are capable of being automated.

In one aspect, the invention provides homogenous methods for determining one or more target mutant sequences in one or more DNA nucleic acid sequences using probes, fluors and fluorescence quenchers. More specifically, a method is provided for amplifying and determining one or more target mutant sequences in a DNA sample, the method comprising the steps of:

(A) forming an admixture comprising

(i) the sample,

(ii) one or more primer pairs specific for said one or more target mutant sequences,

(iii) at least four different nucleoside triphosphates,

(iv) one or more thermostable polymerases,

(v) at least one thermostable restriction endonuclease that is capable of directly cleaving a wild type sequence or wild type sequences of said one or more target mutant sequences or cleaving a primer induced cleavage site, or both,

vi) one or more oligonucleotides comprising one or more fluorescence moieties and one or more fluorescence quenching moieties, said one or more oligonucleotides being capable of hybridizing to DNA comprising said one or more target mutant sequences and capable of producing detectable fluorescence when hybridized thereto;

(B) subjecting the admixture to one or more cycles of heating and cooling, thereby amplifying DNA comprising said one or more target mutant sequences; and

(C) detecting the fluorescence.

In another aspect, the invention provides one or more oligonucleotides comprising a sequence selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67. Any of the oligonucleotides may comprise one or more fluorescence moieties and one or more fluorescence quenching moieties. In particular, SEQ ID NO:14, SEQ ID NO:15, SEQ ID NO:16, SEQ ID NO:17, NO:19, SEQ ID NO:20, SEQ ID NO:21, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67 may comprise one or more fluorescence moieties and one or more fluorescence quenching moieties.

The invention also relates to a method for amplifying DNA comprising a mutant ras sequence in a sample comprising the steps of:

(A) forming an admixture comprising

(i) the sample,

(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,

(iii) at least four different nucleoside triphosphates, one or more thermostable polymerases, and at least one thermostable restriction endonuclease that is capable of directly cleaving wild type K-, H-, or N-ras sequence or cleaving a primer induced cleavage site, or both; and

(B) subjecting the admixture to one or more cycles of heating and cooling thereby amplifying DNA comprising a mutant ras sequence.

The invention also relates to a method for determining one or more ras mutations in a DNA sample comprising the steps of:

(A) forming an admixture comprising

(i) the sample,

(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,

(iii) at least four different nucleoside triphosphates, one or more thermostable polymerases, and at least one thermostable restriction endonuclease that is capable of directly cleaving wild type K-, H-, or N-ras sequence or cleaving a primer induced cleavage site, or both; and

(B) subjecting the admixture to one or more cycles of heating and cooling, thereby amplifying DNA comprising a mutant ras sequence;

(C) separating the DNA by electrophoresis; and

(D) detecting the DNA comprising a mutant ras sequence separated by electrophoresis in step (C)

The invention also relates to a method for determining one or more ras mutations in a DNA sample comprising the steps of

(A) forming an admixture comprising

(i) the sample,

(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,

(iii) at least four different nucleoside triphosphates, one or more thermostable polymerases, and at least one thermostable restriction endonuclease that is capable of directly cleaving wild type K-, H-, or N-ras sequence or cleaving a primer induced cleavage site, or both; and

(B) subjecting the admixture to one or more cycles of heating and cooling, thereby amplifying DNA comprising a mutant ras sequence;

(C) combining the admixture comprising amplified DNA with one or more immobilized oligonucleotides or one or more oligonucleotides capable of being immobilized, said one or more oligonucleotides being capable of hybridizing to DNA comprising a mutant ras sequence thereby capturing DNA comprising a mutant ras sequence; and

(D) detecting the captured DNA comprising a mutant ras sequence.

The sequence of the one or more immobilized oligonucleotides or one or more oligonucleotides capable of being immobilized in any composition, method or kit of the invention may be selected from SEQ ID NO:7, SEQ ID NO:8, or SEQ ID NO:9.

The invention also relates to a method for determining one or more ras mutations in a DNA sample comprising the steps of:

(A) forming an admixture comprising

(i) the sample,

(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,

(iii) at least four different nucleoside triphosphates, one or more thermostable polymerases, and at least one thermostable restriction endonuclease that is capable of directly cleaving wild type K-, H-, or N-ras sequence or cleaving a primer induced cleavage site, or both and,

(iv) one or more oligonucleotides comprising one or more fluorescence moieties and one or more fluorescence quenching moieties, said one or more oligonucleotides being capable of hybridizing to DNA comprising a mutant ras sequence and capable of producing detectable fluorescence when hybridized thereto;

(B) subjecting the admixture to one or more cycles of heating and cooling, thereby amplifying DNA comprising a mutant ras sequence;

(C) detecting the fluorescence.

The invention also relates to a method for amplifying DNA comprising a mutant ras sequence in a sample comprising the steps of:

(A) forming an admixture comprising

(i) the sample,

(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,

(iii) at least four different nucleoside triphosphates and one or more thermostable polymerases;

(B) subjecting the admixture to one or more cycles of heating and cooling;

(C) combining the admixture with one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, provided at least one of the primers is different from the primers in step (A);

(D) subjecting the admixture to one or more cycles of heating and cooling thereby amplifying DNA comprising a mutant ras sequence;

(E) combining the admixture with at least one restriction endonuclease that is capable of directly cleaving wild type K-, H-, or N-ras sequence or cleaving a primer induced cleavage site, or both.

The invention also relates to a method for determining one or more ras mutations in a DNA sample comprising the steps of:

(A) forming an admixture comprising

(i) the sample,

(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,

(iii) at least four different nucleoside triphosphates and one or more thermostable polymerases;

(B) subjecting the admixture to one or more cycles of heating and cooling;

(C) combining the admixture produced after step (B) is performed with one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:36, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, provided at least one of the primers is different from the primers in step (A);

(D) subjecting the admixture to one or more cycles of heating and cooling thereby amplifying DNA comprising a mutant ras sequence;

(E) combining the admixture with at least one restriction endonuclease that is capable of directly cleaving wild type K-, H-, or N-ras sequence or cleaving a primer induced cleavage site;

(F) separating the DNA by electrophoresis; and

(G) detecting the DNA comprising a mutant ras sequence separated by electrophoresis in step (F).

The invention also relates to a method for determining one or more ras mutations in a DNA sample comprising the steps of:

(A) forming an admixture comprising

(i) the sample,

(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,

(iii) at least four different nucleoside triphosphates and one or more thermostable polymerases;

(B) subjecting the admixture to one or more cycles of heating and cooling;

(C) combining the admixture produced after step (B) is performed with one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:11, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, provided at least one of the primers is different from the primers in step (A);

(D) subjecting the admixture to one or more cycles of heating and cooling thereby amplifying DNA comprising a mutant ras sequence;

(E) combining the admixture with at least one restriction endonuclease that is capable of directly cleaving wild type K-, H-, or N-ras sequence or cleaving a primer induced cleavage site, or both;

(F) combining the admixture comprising amplified DNA with one or more immobilized oligonucleotides or oligonucleotides capable of being immobilized, said oligonucleotides. being capable of hybridizing to DNA comprising a mutant ras sequence thereby capturing DNA comprising a mutant ras sequence; and

(G) detecting the captured DNA comprising a mutant ras sequence.

The invention also relates to a method for determining one or more ras mutations in a DNA sample comprising the steps of:

(A) forming an admixture comprising

(i) the sample,

(ii) one or more primer pairs selected from SEQ ID NO:5, SEQ ID NQ:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,

(iii) at least four different nucleoside triphosphates and one or more thermostable polymerases, and

(iv) one or more oligonucleotides comprising one or more fluorescence moieties and one or more fluorescence quenching moieties, said one or more oligonucleotides being capable of hybridizing to DNA comprising a mutant ras sequence and capable of producing detectable fluorescence when hybridized thereto;

(B) subjecting the admixture to one or more cycles of heating and cooling;

(C) combining the admixture produced in step (B) with one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, provided at least one of the primers is different from the primers in step (A);

(D) subjecting the admixture to one or more cycles of heating and cooling thereby amplifying DNA comprising a mutant ras sequence;

(E) combining the admixture with at least one restriction endonuclease that is capable of directly cleaving wild type K-, H-, or N-ras sequence or cleaving a primer induced cleavage site, or both; and

(F) detecting the fluorescence.

In yet another aspect, the invention relates to kits comprising in one or more containers:

(i) one or more oligonucleotides comprising a sequence selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, or SEQ ID NO:55;

(ii) one or more oligonucleotides selected from SEQ ID NO:19, SEQ ID NO:20, SEQ ID NO:21, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, the oligonucleotide or oligonucleotides comprising one or more fluorescence moieties and one or more fluorescence quenching moieties.

The kits may comprise further in one or more containers:

(i) one or more nucleoside triphosphates;

(ii) one or more restriction endonucleases, said restriction endonuclease or restriction endonucleases being capable of directly cleaving wild type K-, H-, or N-ras sequence or a primer induced cleavage site, or both; and

(iii) one or more thermostable polymerases.

In all instances described above, the one or more fluorescence moieties may be linked to one or more nucleotides adjacent to the 3′ terminal nucleotide or linked to the 3′ terminal nucleotide or both and the one or more fluorescence quenching moieties may be linked to one or more nucleotides adjacent to the 5′ terminal nucleotide or the one or more fluorescence quenching moieties may be linked to the 5′ terminal nucleotide or both, and one or more nucleotides comprising the 3′ terminus are complementary to one or more nucleotides comprising the 5′ terminus, or the one or more fluorescence moieties may be linked to one or more nucleotides adjacent to the 5′ terminal nucleotide or linked to the 5′ terminal nucleotide or both, and the one or more fluorescence quenching moieties may be linked to one or more nucleotides adjacent to the 3′ terminal nucleotide or linked to the 3′ terminal nucleotide or both, and one or more nucleotides comprising the 3′ terminus are complementary to one or more nucleotides comprising the 5′ terminus. For example, the fluorescence moieties may be selected from carboxyfluorescein, carboxy-4′,5′-dichloro-2′,7′-dimethoxyfluorescein, tetrachlorofluorescein, dimethoxyfluorescein, or carboxyrhodamine and the quenching moiety may be (4-(4′dimethylaminophenylazo)benzoic acid) or 4(dimethylamine)azobenzene sulfonic acid.

### BRIEF DESCRIPTION OF THE DRAWINGS

FIG. 1 shows a nested PCR/RFLP analysis for K-12 ras mutations in cell lines. K562 cell line DNA: before, lane 1, and after, lane 2, restriction enzyme digestion. Calul cell line DNA: before, lane 3, and after, lane 4, restriction enzyme digestion.

FIG. 2 shows a nested PCR/RFLP analysis for K-12 ras in patient samples; before (lanes 2, 4, and 6) and after (lanes 3, 5, and 7) Bstn1 restriction enzyme digestion.

### DETAILED DESCRIPTION

REMS-PCR (Roberts N. J. et al., 1999, BioTechniques 27:(3)418-422; Ward, R. et al., 1998, Am. J. Pathol 153(2):373-379; WO 96/32500; Fuery, C. J. et al., 2000, Clin. Chem. 46 (5) 620-624), employed in various embodiments of the present invention, utilizes a thermostable restriction enzyme and appropriately designed primers; during PCR thermocycling, wild type sequences and/or primer induced sites are cleaved and mutant sequences are enriched.

In addition to simplifying and reducing the time required for detecting mutations, the invention enables detection of a mutation in the presence of a large excess of wild type DNA (1000-fold and greater).

The invention is described below in detail in examples 1-4, using the restriction enzyme BstN I, which is particularly useful for determining K-ras mutations at codon 12. The invention can be practiced as a tool for analysis following either REMS-PCR or multiple rounds of nested PCR based on digestion with other restriction enzymes including, but not limited to, Bsl I, Msc I, Mse I, Msp I, Bfa I, and Hae III which are useful for determining K-ras mutations at codon 12 (abbreviated K-12, analogous abbreviations are used for the other ras codons), K-13, K-61, H-12, H-13, N-12, N-13, N-61 and mutations at H-ras intron D. Examples 5-7 provide specific sequences of primers and probes that were used for determining such mutations in REMS-PCR methods, methods involving nested PCR followed by restriction endonuclease digestion, and REMS-PCR methods employing molecular beacons.

It was found that a nested PCR method was particularly advantageous for determining ras mutations in samples having low levels of target DNA approaching a single copy in the amplification reaction admixture. This method involved using a primer design such that wild type K-ras sequences were cleaved in an overnight restriction enzyme digestion after nested PCR amplification. Puig et al. 2000, Int. J. Cancer. 85:73-77 describe nested PCR methods for determining K-ras mutations.

Primary tumor samples from individuals were examined for the presence of K-12 ras mutation using molecular beacons. Molecular beacons, described in U.S. Pat. Nos. 5,118,801; 5,312,728 and 5,925,517, are particularly useful in REMS-PCR for automating product detection and for quantifying product. Molecular beacons are oligonucleotide probes that can report the presence of specific nucleic acids using homogeneous methods. They are useful in situations where it is either not possible or desirable to isolate the probe-target hybrids from an excess of the hybridization probes, such as in real-time monitoring of polymerase chain reactions in sealed tubes or in detection of RNAs within living cells. Molecular beacons are hairpin-shaped molecules with an internally quenched fluorophore whose fluorescence is restored when they bind to a target nucleic acid. They are designed in such a way that the loop portion of the molecule is a probe sequence complementary to a target nucleic acid molecule. The stem is formed by the annealing of complementary arm sequences on the ends of the probe sequence. A fluorescent moiety is attached to the end of one arm and a quenching moiety is attached to the end of the other arm. The stem keeps these two moieties in close proximity to each other, causing the fluorescence of the fluorophore to be quenched by energy transfer. Since the quencher moiety is a non-fluorescent compound and emits the energy that it receives from the fluorophore as heat, the probe is unable to fluoresce. When the probe encounters a target molecule, it forms a hybrid that is longer and more stable than the stem and its rigidity and length preclude the simultaneous existence of the stem hybrid. Thus, the molecular beacon undergoes a spontaneous conformational reorganization that forces the stem apart, and causes the fluorophore and the quencher to move away from each other, leading to the restoration of fluorescence which can be detected. In order to detect multiple targets in the same solution, molecular beacons can be made in many different colors utilizing a broad range of fluorophores (Tyagi, S. et al., 1998, Nature Biotechnology, 16, 49-53). DABCYL, a non-fluorescent compound, can serve as a universal quencher for any fluorophore in molecular beacons.

Sectioning and DNA Extraction

Except where noted, all reagents used in protocols described in this disclosure were purchased from Sigma-Aldrich (St. Louis, Mo.), and all oligonucleotides were synthesized at Ortho-Clinical Diagnostics.

Using a microtome, paraffin blocks comprising tumor or lymph node tissue were sectioned: 10 microns thick for primary tumor samples, and 50 microns thick for lymph node samples. To avoid DNA contamination between samples, excess paraffin was removed from the microtome before the first section was cut. All excess paraffin was removed by brush, and the blade area was wiped with xylene and allowed to air dry prior to use. A fresh blade was used between patient sample paraffin blocks. After cutting, sections were carefully transferred into separate 1.5 mL conical screw-cap tubes by means of a wooden applicator stick. A new stick was used for each paraffin block. In control experiments it was shown that this method successfully eliminated carryover between K-12 ras-positive and K-12 ras-negative samples as determined by REMS-PCR.

To extract DNA, the tubes were centrifuged at 14,000 rpm for 2 min to pellet the paraffin, and 80 microliters of lysis buffer (10 mM Tris-HCl, pH 8.0, and 0.5% Tween 20 and 10 microliters of PreTaq (Life Technologies, Inc., Gaithersburg, MD.) were added and the tube was incubated at 100° C. in a heat block for 5 min. Ten microliters of 250 mM sodium hydroxide was added and the tubes were incubated in a heat block at 105° C. for 10 min. While hot, the tubes were centrifuged at 14,000 rpm for 2 min. The liquid under the paraffin layer was carefully removed, transferred to a new tube, and stored frozen prior to use.

Cell-Lines

All human cell lines were purchased from the American Type Culture Collection, Manassas, Va. Calu 1 (ATCC HTB54) is a cell line derived from a lung adenocarcinoma which is heterozygous at K-ras codon 12 having both a wild type (GGT) and a mutant (TGT) sequence(5). K562 (ATCC CCL243) is a cell line derived from a human leukemia, which is wild type at codon 12 of K-ras (Roberts, N. J. et al., 1999, BioTechniques 27:(3)418-422). Other human cell lines known to have ras mutations used include Molt-4 (ATCC CRL-1582) having an N-ras mutation at codon 12(Bos, J. L., 1988, Hematol. Pathol. 2:55-63), HCT-116 (ATCC CCL-247) having a K-ras mutation at codon 13 (Aoki, T., S. et al., 1994, Hum. Mutat.:3(4):342-6), HL-60 (ATCC CCL-240) having an N-ras at codon 61 (Bos, J. L. et al., 1984, Nucleic Acids Res. 12(23):9155-63) and T24 (ATTCC HTB-4) having an H-ras mutation at codon 12 (Capon, D. J. et al., 1983, Nature 302: 33-37) and intron D (Cohen,. J. N. and A. D. Levinson, 1988,. Nature 334 (6178): 119-124). Genomic DNA was extracted from cell lines using a protocol involving incubation of cells in a lysis buffer at high temperature.

Oligonucleotides Having Mutant Sequences

In some experiments oligonucleotides prepared according to the method of Rochlitz et al. (1988, DNA 7(7):515-519) were used as target nucleic acids (identified as “oligo” in Table 2). They comprised the base sequence of N-ras codon 13 having a cytosine to thymine (C to T) mutation.

Definitions

When reference is made to a nucleotide “adjacent” to a terminal nucleotide in an oligonucleotide comprising a particular sequence of interest, the term “adjacent” means any nucleotide between the terminal nucleotide and the first nucleotide commencing the sequence of interest. For example, consider an oligonucleotide having the following hypothetical 5′ to 3′ directed sequence:

AGTCGTTAGTGTCATCTATAGAGACTCGGGCCTGACTAG

The underlined sequence CATCTATAGAGA represents the particular sequence of interest, the 5′ terminal nucleotide (in bold font) is A and the 3′ terminal nucleotide (in bold font) is G. A nucleotide adjacent to the 5′ terminal nucleotide is any nucleotide between A and C, where C is the first nucleotide commencing the sequence of interest in the 5′ to 3′ direction. That is, it is any nucleotide in the sequence GTCGTTAGTGT. Analogously, a nucleotide adjacent to the 3′ terminal nucleotide is any nucleotide in the sequence CTCGGGCCTGACTA.

When reference is made to the “3′ terminus” it means the nucleotides comprising those adjacent to the 3′ terminal nucleotide, as defined above, and the 3′ terminal nucleotide. An analogous definition applies to the “5′ terminus”.

An oligonucleotide comprising a sequence of interest can be obtained or prepared from a natural source or prepared by way of any suitable chemical synthetic method. An oligonucleotide can consist of only the sequence of interest, or the sequence of interest itself may be only part of a larger sequence of nucleotides comprising the oligonucleotide. An oligonucleotide may have linked to it by way of one or more nucleotides, any molecule or molecules in addition to a nucleotide, such as a linker molecule for covalent bonding to another molecule or substrate; a label, such as a fluor, dye, radioisotope or enzyme; a molecule that interacts with another molecule which may or may not also be linked to the oligonucleotide, such as a fluorescence quencher; a ligand for binding to a specific receptor, such as biotin, avidin or strepavidin, and so forth.

As used herein the term “wild type sequence” refers to a conserved sequence of nucleotides within a gene in a biological species, preferably a human gene, that is, a sequence that is observed in a majority of representative members of the species.

The term “primer” refers to an oligonucleotide, whether naturally occurring or synthetically produced, that is capable of acting as a point of initiation of synthesis when placed under conditions in which synthesis of a primer extension product complementary to a nucleic acid strand (that is, template) is induced. Primers may be perfectly matched to the target sequence or they may contain internal mismatched bases, which can result in the induction of restriction endonuclease recognition/cleavage sites in specific target sequences. For the purpose of the present invention, this shall be referred to as a primer induced recognition/cleavage site. Any number of primers capable of inducing a cleavage site in one or more target sequences can be used in a reaction admixture and the statement “a primer induced cleavage site” is meant to include a single primer induced site or multiple primers inducing different cleavage sites in one or more target sequence.

The term “primer pair” refers to two primers, one being capable of acting as a point of initiation of synthesis of a primer extension product on one strand of a duplex DNA target or on one strand derived from a duplex DNA target, and the other primer being capable of acting as a point of initiation of synthesis of a primer extension product on the other strand of a duplex DNA target or on the other strand derived from the duplex DNA target.

### EXAMPLE 1

Detection of Ras Mutations in Primary Tumor Samples Using REMS-PCR

Mutations in codon 12 of the K-ras gene were detected using REMS-PCR according to the methods of Roberts, N. J. et al., 1999, BioTechniques 27:(3)418-422; Ward, R. et al., 1998, Am. J. Pathol. 153(2):373-379; and WO 96/32500. Each PCR admixture contained three pairs of primers. The diagnostic primers induce a BstN I restriction site in the wild type ras, but not in a ras mutation at codon 12. Thus, ras wild type DNA is selectively cleaved during PCR thermocycling, and mutant sequences of ras at codon 12 are enriched. The PCR control primers were used to confirm that PCR amplifiable DNA was extracted, and the enzyme control primers confirmed that the restriction enzyme was functioning during thermocycling. Reaction admixtures contained 12 units/100 μL of recombinant Taq polymerase (developed at Ortho-Clinical Diagnostics), and a 5-fold molar excess (0.842 μL) of Taq inhibiting antibody TP4-9.2 (developed at Ortho-Clinical Diagnostics according to protocols described in U.S. Pat. Nos. 5,338,671 and 5,587,287) over the polymerase, HT50 buffer (100 mM sodium chloride, and 50 mM Tris-HCl , pH 8.3), 0.3 μM of diagnostic primers (see below), 5BKIT (SEQ ID NO: 1), (Roberts, N. J. et al., 1999, BioTechniques 27:(3)418-422; Ward, R. et al., 1998, Am. J. Pathol. 153(2):373-379) and 3K2 (SEQ ID NO:2) (Ward, R. et al., 1998, Am. J. Pathol. 153(2):373-379), 0.05 μM of PCR control primer pairs, 5BK5 (SEQ ID NO:3) and 3K6 (SEQ ID NO:4), 0.05 μM of enzyme control primer pairs, 5BK28 (SEQ ID NO:5) and 3K29 (SEQ ID NO:6), 0.2 mM total dinucleoside triphosphates (dNTPs), 0.6 units/μL of BstN I (New England BioLabs, Beverly Mass.), 1 mM dithiothreitol (DTT), 4 mM magnesium chloride, sample (typically 3 μRL) and deionized water up to a final volume of 100 μL. At least one of the primers used in Example 1 was biotinylated. Biotinylated primers, after extension by polymerase, are captured using avidin reagents to generate signal. In all the examples, the letter “B” appearing in the name of an oligonucleotide identifies it as being biotinylated. If the oligonucleotide is not biotinylated, the letter “B” does not appear in the name identifying it. For example, a primer having sequence SEQ ID NO: 5, if biotinylated, it was named 5BK28, if it is not biotinylated, it was named 5K28. Biotinylated and non-biotinylated primers provide substantially the same results as determined using gel-based detection. The following primers were biotinylated at the 5′ end: 5BKIT (SEQ ID NO: 1), 5BK5 (SEQ ID NO: 3), 5BK28 (SEQ ID NO: 5), 5BKITSC (SEQ ID NO: 12), 5BN2 (SEQ ID NO: 28), 5BN4 (SEQ ID NO: 29), 3BN13 (SEQ ID NO: 55), 5BK15 (SEQ ID NO: 60), and 5BK37 (SEQ ID NO: 61). The nucleotide sequences of the primers are as follows:

| | |
|---|---|
| TATAAACTTG TGGTAGTTGG ACCT | SEQ ID NO: 1 |
| CGTCCACAAA ATGATTCTGA | SEQ ID NO: 2 |
| TCAGCAAAGA CAAGACAGGTA | SEQ ID NO: 3 |
| AGCAATGCCC TCTCAAGA | SEQ ID NO: 4 |
| AGTAAAAGGT GCACTGTAAT AATC | SEQ ID NO: 5 |
| GTGTCGAGAA TATCCAAGAG CCA | SEQ ID NO: 6 |

The solutions comprising Taq polymerase and anti-Taq antibody were combined and incubated for 10-15 minutes prior to the addition of the other PCR components. BstN I restriction enzyme was added to the reaction admixture just prior to the addition of sample, the last component added.

In the case of primary tumor samples, the reaction admixture was amplified and detected using an Ortho-Clinical Diagnostics, Inc. pouch containment system for nucleic acid amplification and detection as described by Findlay et al. (1993, Clin. Chem. 39(9):1927-1933) and U.S. Pat. No. 5,229,297. Sample (approximately 0.8 ug DNA), combined with PCR reagents to a total volume of 85 ul as described above, was loaded into a blister of the pouch and the pouch was sealed. The PCR blister was heated for 1 min at 94° C., followed by 30 PCR cycles (a melt temperature of 94° C. for 10 sec, followed by an annealing temperature of 58° C. for 75 seconds). After a post-amplification incubation for 5 min at 103° C., product was detected in a “detection” blister of the pouch, wherein product hybridized with complementary oligonucleotide (capture oligo) attached to beads. The method of oligo attachment to polystyrene beads has been described by Findlay et al. (1993, Clin. Chem. 39 (9):1927-1933) and in U.S. Pat. No. 5,380,489 and involves the use of polymeric particles (1.2 um average diameter) of poly(styrene-co-mono-2(p-vinylbenzylthio)ethyl succinate (95:5 weight ratio) prepared by known emulsion polymerization techniques. To these particles were attached molecules of the indicated capture oligo K-CapD-8 (SEQ ID NO:7), Cap-2E (SEQ ID NO:8), or K-Cap6 (SEQ ID NO: 9). Attachment was though an aminediol linking group with two tetraethylene glycol spacer groups prepared and attached to the oligonucleotide according to the teaching of U.S. Pat. No. 4,962,029. The oligonucleotide molecules were attached to the particles to form a nucleic acid reagent as described in U.S. Pat. No. 5,380,489, example 3.

Capture oligo, K-CapD8 (SEQ ID NO:7), hybridized to biotinylated REMS-PCR diagnostic product, capture oligo, Cap-2E (SEQ ID NO:8), hybridized to biotinylated enzyme contol PCR product, and capture oligo, K-Cap6 (SEQ ID NO: 9), hybridized to biotinylated PCR control product. The nucleotide sequences follow:

| | |
|---|---|
| TATCGTCAAG GCACTCTTGC CTACGCCA | SEQ ID NO: 7 |
| GACTGTGTTT CTCCCTTCTC AGGATTCC | SEQ ID NO: 8 |
| GACATAACAG TTATGATTTT GCAGAAAACA GATC | SEQ ID NO: 9 |

The horseradish peroxidase (HRP) channel and wash channel of the pouch were at 55° C. The detection channel was at 40° C. Hybridized product was detected using a solution of HRP-streptavidin and HRP-dye substrate. Each pouch contained 3 detection blisters, each with 200 μL of reagent solution (streptavidin-HRP, 200 μL/blister; wash 200 μL/blister; and dye/gel 200 μL/blister). The order in which the blisters were used was as follows: HRP-streptavidin, wash, and finally dye/gel blister. The capture oligo beads were ordered in the pouch (in the direction of reagent flow) as follows: no beads, K-Cap2E, no beads, K-capD8, no beads, K-Cap6M, and no beads.

Using the REMS-PCR based method in a pouch format as described, approximately 51% of the primary tumor samples from 106 Dukes' B colon cancer patients were found to possess a K-ras mutation at codon 12. Forty-five of the samples (49%) were negative for K-ras mutation at codon 12. In comparison, in a multi-center study (Andreyev, H. J. N. et al, 1998, Natl. Cancer Inst. 90 (9):675-684) it was reported that 43.8% of colon cancer primary tumors, as determined by single-stranded conformation polymorphism (SSCP) techniques, exhibited a K-12 ras mutation, 33.1% by direct sequencing, and 32.7% by allele-specific primer PCR. The greater incidence of K-12 ras mutation observed using the methods of the present invention may be due to differences in the ability of each method to detect a mutation in a large excess of wild type DNA. Also, increased sensitivity is possible because product can be detected using enzyme-mediated calorimetric, florescence, or chemiluminescense signal formation methods.

The REMS-PCR based method is sensitive, utilizes internal PCR and enzyme controls, and is rapid. The method was carried out in a pouch containment device, which allowed automation. The method reduced the possibility of contamination, and permitted increased detection sensitivity, as product was detected using enzyme-catalyzed dye formation, in contrast with detection of product in a gel subsequent to electrophoresis (Findlay, J. B. et al., 1993, Clin. Chem. 39(9):1927-1933). The method, subsequent to DNA extraction, took less than 90 minutes to complete.

### EXAMPLE 2

Detection of K12-ras Mutations in Lymph Node Samples Using Nested PCR

Lymph nodes from a total of 38 Dukes' B colorectal cancer patients, identified as having a K-12 ras mutation in the primary tumor, were analyzed for the presence of K-12 ras mutations. Because of the small amount of lymph node tissue, and therefore nucleic acids, in the paraffin blocks and the limited stability of the BstN I restriction enzyme during thermocycling, which permitted only about 34 amplification cycles, the REMS-PCR method described based on BstN I above for primary tumor samples was less sensitive than desired for amplification and detection of low copy number samples. Therefore, a more sensitive nested PCR method involving two separate rounds of amplification, followed by restriction endonuclease digestion was used.

Extended PCR thermocycling in this method (12 cycles in round 1 and 40 cycles in round 2) allowed greater sensitivity for low copy number samples. For the present experiments, a Stratagene Eagle Eye II Still Video System (Stratagene, La Jolla, Calif.) was used. This instrument uses sensitive CCD optics and video and software enhancement to improve resolution of gel bands.

Round 1 PCR admixtures contained 4 units/50 μL of recombinant Taq polymerase, a 5-fold molar excess of Taq inhibiting antibody TP1-12.2, Cetus II buffer (50 mM KCl in 10 mM Tris-HCl , pH8.3), 1.2 μM each of primers 5KID (SEQ ID NO:10 and 3KiE (SEQ ID NO:11), 0.2 mM total dinucleoside triphosphates (dNTPs), 0.04 mM magnesium chloride, sample (typically 5 μL) and deionized water up to a final volume of 50 μL. Taq poymerase and anti-Taq antibody were combined and incubated for 10-15 minutes prior to the addition of the other PCR components. Thermocycling was performed in a Gene-Amp 9600 (Perkin-Elmer, Norwalk, Conn.) with the following parameters for Round 1: An initial incubation at 94° C. for 3 min, followed by 12 cycles of alternate incubations at 94° C. for 10 sec, and 60° C. for 30 sec. The primer sequences are as follows:

| | |
|---|---|
| GGCCTGCTGA AAATGACTGA ATA | SEQ ID NO: 10 |
| CTCATGAAAA TGGTCAGAGA AAC | SEQ ID NO: 11 |

Round 2 PCR admixtures contained 10 units/100 μL of recombinant Taq polymerase, a 5-fold molar excess of Taq inhibiting antibody TP12.2, HT50 buffer (100 mM sodium chloride, and 50 mM Tris-HCl, pH 8.3), 0.2 μM each of primers 5BKITSC (SEQ ID NO:12) and 3KiU (SEQ ID NO:13) (see below), 0.2 mM total dNTPs, 0.04 mM magnesium chloride, 2 μL sample, and deionized water up to a final volume of 100 μL. Round 2 thermocycling parameters performed on a Gene-Amp 9600 were: An initial incubation at 94° C. for 3 min, followed by 40 cycles of alternate incubations at 94° C. for 10 sec and 60° C. for 30 sec. The primer sequences are as follows:

| | |
|---|---|
| GAATATAAAC TTGTGGTAGT TGGACCT | SEQ ID NO: 12 |
| ATCAAAGAAT GGTCCTGCACC | SEQ ID NO: 13 |

Restriction enzyme digestion was performed by combining 40 units of BstN I, (New England BioLabs, Beverley Mass.), and 15 uL of the amplification product from Round 2 in a microfuge tube. Tubes were incubated overnight at 60° C.

Samples were analyzed by electrophoresis on 4% w/v NUSieve agarose gel (FMC Bioproducts, Rockland, Me.) and imaged by means of a Stratagene Eagle Eye II video system (La Jolla, Calif.).

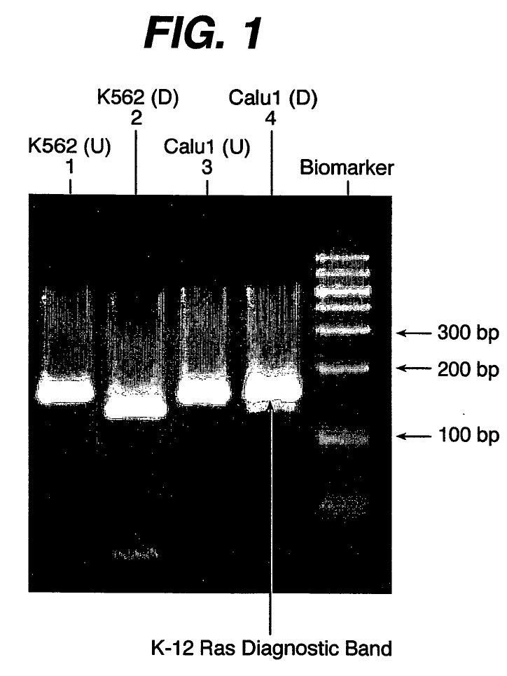

FIG. 1 shows the results obtained with K562 cell line DNA, which is wild type for K-ras, before (lane 1) and after (lane 2) restriction enzyme digestion. Calul DNA, heterozygous for a K-ras mutation at codon 12 is shown before (lane 3) and after (lane 4) restriction enzyme digestion. Thus, a gel band at 152 bp remaining after BstN I digestion is diagnostic for a K-ras mutation at codon 12, whereas gel bands of 128 bp and 24 bp are formed as a result of Bstn1 digestion of the K-ras wild type product.

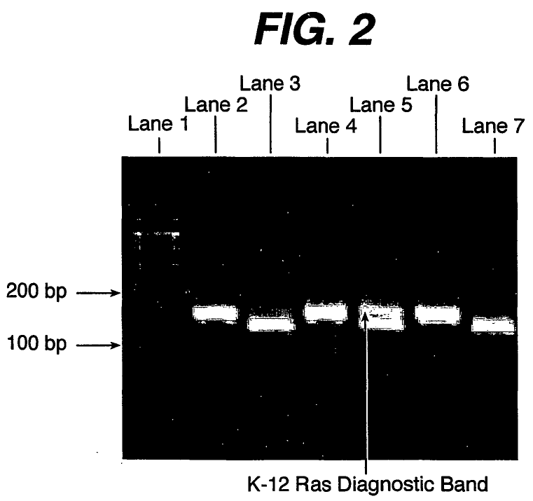

FIG. 2 shows results obtained for both K-12 ras positive and negative lymph nodes, as assayed by the nested PCR protocol followed by Restriction Fragment Length Polymorphism (RFLP) gel analysis. In FIG. 2, results for three different lymph node samples before (lanes 2, 4, and 6) and after BstN I restriction enzyme digesion (lanes 3,5 and 7) are shown. The 152 bp product remaining after restriction enzyme digestion (lane 5) is diagnostic for a K-ras mutation at codon 12, and the absence of a 152 bp product after digestion is diagnostic for wild type K-ras at codon 12 (lanes 3 and 7). The lymph node samples in lanes 2 and 3, and in lanes 6 and 7 are negative for a K-12 ras mutation and the lymph node sample in lanes 4 and 5 are positive for this mutation.

Of the 38 samples, 14 (37%) were positive for a K-12 ras mutation in one or more lymph nodes, whereas 24 samples (63%) were negative for a ras mutation. For the 14 lymph node samples exhibiting a K-12 ras mutation, a total of 142 nodes were evaluated (an average of 10 nodes per patient). Similarly, for the 24 samples that were negative for a K-12 ras mutation in one or more lymph nodes, a total of 97 nodes were evaluated (an average of 4 nodes per patient).

In the REMS-PCR and nested PCR based methods one or more of the following “negative” controls were used: (a) a tonsil sample prepared from a paraffin block as described in Example 1, (b) genomic DNA from cell line K562 (K-ras wild type), and (c) a reagent control in which no DNA template was added. Genomic DNA from the cell line Calu-1 was used as a “positive” control. Genomic DNA was prepared as described previously in the present application.

### EXAMPLE 3

Detection of K-12 Ras Mutations Using Molecular Beacons in REMS-PCR

Samples, reaction mixtures, and primers were as described in example 1 except that 0.2 μM of the diagnostic primers were used. To each of three microtiter wells was added 1 μL of a 20 μM stock of the molecular beacons. Molecular beacon synthesis was performed at Synthetic Genetics, San Diego Calif. Each molecular beacon was labeled with the dye carboxyfluorescein, FAM, on the 5′ end and the fluorescence quencher DABCYL (4-(4′-dimethylaminophenylazo)benzoic acid) on the 3′ end. Typically the starting material for the synthesis of molecular beacons is an oligonucleotide that contains a sulfhydryl group at its 5′-end and a primary amino group at its 3′-end. DABCYL is coupled to the primary amino group utilizing an amine-reactive derivative of DABCYL. The oligonucleotides that are coupled to DABCYL are then purified. The protective trityl moiety is then removed from the 5′-sulfhydryl group and a fluorophore is introduced in its place using an iodoacetamide derivative. Recently a control pore column that can introduce DABCYL moiety at the 3′ end of an oligonucleotide has become available which makes it possible to synthesize a molecular beacon completely on a DNA synthesizer.

The probe sequences are as follows:

SEQ ID NO: 14

GCGAGCTATC GTCAAGGCAC TCTTGCCTAC GCCAGCTCGC

SEQ ID NO: 15

CCGAGCGACA TAACAGTTAT GATTTTGCAG AAAACAGAGC TCGG

SEQ ID NO: 16

GCGAGAAGC CTTCGCCTGT CCTCATGTAT TGGTGCTCGC

SEQ ID NO: 17

GCGAGCGACT GTGTTTCTCC CTTCTCAGGA TTCCGCTCGC

For each analysis three separate microtiter wells were used. BD2 (SEQ ID NO:14) was added to a first well as a mutant K-12 ras diagnostic probe; increased fluorescence signal indicating a K-ras mutation at codon 12. A second well contained BPIG (SEQ ID NO:15) as a PCR control; increased fluorescence indicates sufficient amplifiable DNA. A third well contained either BE1 (SEQ ID NO:16) or BE2 (SEQ ID NO:17) as an enzyme control. Increased fluorescence of the enzyme control would indicate that the restriction enzyme may have been inactivated during PCR thermocycling, and would represent a failed assay.

Thermocycling and fluorescence detection were carried out using an ABI Prism 7700 Sequence Detector (PE Applied Biosystems, Foster City, Calif.). Thermocycling parameters were: A first incubation at 50° C. for 1 min, a second incubation at 94° C. for 1 min, followed by 40 cycles of alternate heating at 94° C. for 10 sec and heating at 58° C. for 75 sec. After the last cycle, the reagent admixture was incubated at 50° C. for 2 min. The single reporter mode was used for detecting fluorescence. Results are summarized in Table 1 below.

For a valid determination, the diagnostic and PCR control must develop an increase in fluorescence signal above a threshold level; whereas, fluorescence of the enzyme control must remain below this threshold level. Cₜ, which is reported in Table 1, is the calculated cycle number at which point the fluorescence signal exceeds the baseline threshold value established during approximately the first 15 PCR cycles (see ABI PRISM 7700, “Sequence Detection System”, User's Manual, 1998, pp D4-D5, Perkin-Elmer Corp, Foster City, Calif.). The smaller the Cₜ value, the earlier amplified product is detected. The Cₜ value is related, therefore, to the presence and amount of target in the sample.

TABLE 1
Molecular Beacon-Based Detection of K-12 ras Mutations

| Well | Beacon | Sample | Ct |
|---|---|---|---|
| B2 | BP1G | 1:10 (C:K) | 25.76 |
| B4 | BD2 | 1:10 (C:K) | 30.01 |
| B6 | BE1 | 1:10 (C:K) | >40.00 |
| A2 | BP1G | 1:10 (C:K) | 25.32 |
| A4 | BD2 | 1:10 (C:K) | 28.01 |
| A6 | BE2 | 1:10 (C:K) | 37.31 |
| B1 | BP1G | K562 | 28.28 |
| B3 | BD2 | K562 | >40.00 |
| B5 | BE1 | K562 | >40.00 |
| A1 | BP1G | K562 | 27.70 |
| A3 | BD2 | K562 | 38.64 |
| A5 | BE2 | K562 | >40.00 |

Replicate determinations for samples containing a 1:10 ratio, by weight, of K-12 ras mutant to wild type DNA (1:10 (C:K)) are shown in Table 1. Duplicate wells with BP1G, the PCR amplification control, had Ct values of 25.76 and 25.32, indicating that the samples contained PCR amplifiable DNA. Duplicate determinations with BD2 resulted in Cₜ values of 30.01 and 28.01, indicating that the samples are positive for a K-12 ras mutation. Cₜ values with enzyme controls BE1 and BE2 were considerably higher at 40.00 and 37.31, respectively, indicating that the restriction enzyme was active during thermocycling.

With wild type K562 cell line DNA alone (K562 in Table 1), duplicates with the BPIG PCR control had Ct values of 28.28 and 27.70, indicating the presence of amplifiable DNA.

The wells containing BD2 had Cₜ values of 40 and 38.2 indicating that the samples were negative for a K-12 ras mutation. The wells containing the enzyme controls BE1 or BE2 had Cₜ values of 40.00.

### EXAMPLE 4

Detection of K-12 ras Mutations Using Molecular Beacons in Multiplexed REMS-PCR Methods

This example illustrates the use of molecular beacons comprising different fluorophores for multiplexed REMS-PCR based determination of K-12 ras mutations.

Each REMS-PCR reaction admixture was as described in example 1, except that primer 5BKITSC (SEQ ID NO:12) was substituted for primer 5BKIT (SEQ IS NO:l) and 0.1 μM enzyme control primers and 0.1 μM PCR control primers were used. Each microtiter well also contained 0.1 μM of molecular beacons BP1-TET (SEQ ID NO:19) and BE6-JOE (SEQ ID NO:21) and 0.20 μM of molecular beacon BD-FAM(SEQ ID NO:20). Molecular beacon, BP1-TET, comprised the fluor tetrachlorofluorescein (TET) at its 5′end. Molecular beacon BD3-FAM comprised the fluor carboxyfluorescein (FAM) at its 5′end. Molecular beacon BE6-JOE comprised the fluor carboxy-4′,5′-dichloro-2′,7′ dimethoxyfluorescein (JOE) at its 5′ end. The quencher DABCYL was attached at the 3′end. Molecular beacons comprising FAM or TET were purchased from Synthetic Genetics, San Diego, Calif., and the JOE-labeled beacon was purchased from Tri-Link BioTechnologies, Inc., San Diego, Calif. The target directed DNA sequences are as follows:

SEQ ID NO: 18

GGATATTCTC GACACAGCAG GTT

SEQ ID NO: 19

GCGAGCGACA TAACAGTTAT GATTTTGCAG AAAACAGATC GCTCGC

SEQ ID NO: 20

GCGAGCCTAT CGTCAAGGCA CTCTTGCCTA CGCCAGCTCG C

SEQ ID NO: 21

GCGAGCAGGA ATCCTGAGAA GGGAGAAACA CAGTCGCTCG C

Thermocycling conditions on the ABI Prism 7700 Sequence Detector were as described in example 3.

For the PCR control beacon, BP1-TET, Cₜ values for duplicate determinations, at a 1:100 weight ratio of mutant to wild type DNA were 31.902 and 31.176, indicating the presence of amplifiable DNA. For the diagnostic beacon, BD3-FAM, at a 1:100 ratio of mutant to wild type DNA, Cₜ values of 35.058 and 36.225 were obtained, indicating the presence of K-12 ras mutation. Ct values for the enzyme control beacon, BE6-JOE, were 40.000 and 40.000, confirming that the BstN I restriction enzyme was active.

These results show that K-12 ras mutations can be determined using molecular beacons in multiplexed REMS-PCR-based methods.

### EXAMPLE 5

Multiplexed Method for Common K-, H-, and N-ras Mutations

For multiplexed detection of ras mutations, a nested PCR protocol involving two rounds of PCR was used. Round 1 PCR reaction admixtures contained MgCl2, DNTP, and Taq polymerase, and anti-Taq antibody TP1.12.2 in the concentrations described in Example 1, as well as 0.2 μM of the indicated Round I primer in Table 2, 0.8 ug DNA, Cetus Buffer II (50 mM KCl and 10 mM Tris-HCl, pH8.3) and water to a final volume of 50 μL. The appropriate wild type and mutant cell line or synthetic DNA were included for each assay as shown in Table 2.

Thermocycling was performed on a Gene-Amp 9600 (Perkin-Elmer, Norwalk, Conn.) with the following parameters for Round 1: 1 cycle of 94° C. for 3 mins, followed by 12 cycles of alternate incubations at 94° C. for 10 sec, and 55° C. for 30 sec. For the assay of mutations in H-ras intron D, 20 cycles of PCR amplification were used instead of 12 cycles.

Round 2 PCR reaction admixtures contained 10 units/100 μL of recombinant Taq polymerase (developed at Ortho-Clinical Diagnostics), and a 5-fold molar excess (0.842 μL) of Taq inhibiting antibody TP1-12.2 over the polymerase, HT50 buffer (100 mM sodium chloride, and 50 mM Tris-HCl , pH 7.5), 0.2 mM total dinucleoside triphosphates (dNTPs), 3 mM magnesium chloride, 0.2 uM of the indicated Round 2 primers in Table 2, DNA sample from Round 1 (typically 3 μL) and deionized water up to a final volume of 100 μL.

Thermocycling was performed on a Gene-Amp 9600 (Perkin-Elmer, Norwalk, Conn.) with the following parameters for Round 2: 1 cycle of 94° C. for 3 mins, followed by 32 cycles of alternate incubations at 94° C. for 10 sec, and 60° C. for 30 sec. For the assay of mutations in H-ras intron D, 38 cycles of PCR amplifcation were used instead of 32 cycles.

Restriction enzyme digestions were prepared by mixing 15 uL of PCR product from Round 2, 2 uL of restriction enzyme buffer (10× stock), the indicated units of each restriction enzyme in Table 2 and water to a final volume of between 17 and 20 uL. Restriction enzyme digestion buffers were purchased from New England BioLabs (Beverly, Mass.). For BstN I, Mse I, Hae III digestions, NEB2 buffer was used, and for Bsl 1 digestions, NEB3 buffer was used. Digestion with all other restriction enzymes shown in Table 2 used NEB4 buffer, except for Mae I, which used SuRE/Cut Buffer purchased from Roche Molecular Biochemicals, Indianapolis, Ind.). Overnight digestion at the temperature indicated in Table 2 was used.

After restriction enzyme digestion, samples were analyzed by electrophoresis on 4% w/v NUSieve agarose gel (FMC Bioproducts, Rockland, Me.) and imaged by means of a Stratagene Eagle Eye II video system (La Jolla, Calif.). As provided in Table 2, a mutation in the particular ras gene is indicated by the presence of a gel band similar to that of undigested product. Wild type DNA is cleaved by the restriction enzyme to two smaller size gel bands of expected molecular weights as provided in Table 2.

The primers having the target directed sequences identified below were used in REMS-PCR and/or nested PCR methods for determining mutations in K-12, K-13, K-61, H-12, H-13, N-12, N-13, N-61 and mutations in H-ras intron D.

| | |
|---|---|
| GTAGTAATTG ATGGAGAAAC CTGT | SEQ ID NO: 22 |
| TGGACATACT GGATACAGCT GGACT | SEQ ID NO: 23 |
| CGGCCCCTCG CGCTTTA | SEQ ID NO: 24 |
| AGCTGTGTCG GCCCAGGACT GCA | SEQ ID NO: 25 |
| ATGTGACCCA GCGGCCCCTC G | SEQ ID NO: 26 |
| CTATAATGGT GAATATCTTC AAATG | SEQ ID NO: 27 |
| AGTACAAACT GGTGGTGCCT GGAG | SEQ ID NO: 28 |
| ACTGGTGGTG GTTCCAGCAG GT | SEQ ID NO: 29 |
| ATATAAACTT GTGGTAGTTC CAGCTGGT | SEQ ID NO: 30 |
| GGTTCTGGAT TAGCTGGATT G | SEQ ID NO: 31 |
| GGATATTCTC GACACAGCAG GC | SEQ ID NO: 32 |
| GGGAGACGTG CCTGTTGGAC | SEQ ID NO: 33 |
| TTGATGGCAA ACACACACAG GA | SEQ ID NO: 34 |
| ACAAGTGGTT ATAGATGGTG AAAC | SEQ ID NO: 35 |
| TGATGGCAAA TACACAGAGG A | SEQ ID NO: 36 |
| GGACATACTG GATACAGCTG GC | SEQ ID NO: 37 |
| TTGGAGATCC TGGATACCGC TGG | SEQ ID NO: 38 |
| CCCTGAGGAG CGATGACGGA A | SEQ ID NO: 39 |
| AGTGGGGTCG TATTCGTCC | SEQ ID NO: 40 |
| TCACCTCTAT AGTGGGGTCG TA | SEQ ID NO: 41 |
| GTTCTTGCTG GTGTGAAATG AC | SEQ ID NO: 42 |
| AGGTCCTTGC TGGTGTGAAA TGACTG | SEQ ID NO: 43 |
| GTGGTTCTGG ATTAGCTGGA TTGTCAG | SEQ ID NO: 44 |
| GTTGGACATA CTGGATACAG CTGGC | SEQ ID NO: 45 |
| GGCAAATACA CAGAGGAAGC CTTCG | SEQ ID:NO 46 |
| GTTGGACATA CTGGATACAG CTGGACT | SEQ ID NO: 47 |

Specific primer sets used in nested PCR and their ras targets are identified in Table 2 below. The restriction enzyme, added subsequent to round 2 amplification, cleaves the wild type gene.

TABLE 2

| | | | | | | | | | Size of | | |
|---|---|---|---|---|---|---|---|---|---|---|---|
| | | | | | | | | Restriction | Round 2 | Size of | Source of |
| | | | | | | | | Digestion | Undigested | Round 2 | Target |
| | | Round 1 | | Round 2 | | Restriction | | Temp | Product, | Digested Product, | Nucleic |
| Assay | Codon | Primers* | | Primers* | | Enzyme | | (° C.) | number of bases | number of bases | Acid |
| 1 | N-13 | 5N1S | (43) | 5BN4 | (29) | 10 | U Bsl 1 | 60 | 67 | 47 and 20 | oligo |
| | | 3N5S | (44) | 3N9S | (46) | | | | | | |
| 2 | N-61 (3) | 5N6 | (35) | 5N61C | (47) | 15 | U Bfa 1 or | 37 | 94 | 68 and 26 | none |
| | | 3N9S | (46) | 3N9S | (46) | 4 | U Mae 1 | | | | |
| 3 | K-12 | 5KID | (10) | 5BKITSC | (12) | 20 | U BstN I | 60 | 152 | 128 and 24 | Calu-1 |
| | | 3KIE | (11) | 3KIU | (13) | | | | | | |
| 3 | N-61 | 5N6 | (35) | 5N7 | (37) | 12 | U Msc I | 37 | 95 | 77 and 18 | HL60 |
| | (1, 2) | 3N9 | (36) | 3N9 | (36) | | | | | | |
| 3 | N-13 | 5N1 | (42) | 5BN4 | (29) | 10 | U Bsl I | 60 | 65 | 46 and 19 | oligo |
| | | 3N5 | (31) | 3N5 | (31) | | | | | | |
| 4 | N-61 | 5N6 | (35) | 5N7 | (37) | 12 | U Msc I | 37 | 94 | 70 and 24 | HL60 |
| | (1, 2) | 3N9S | (46) | 3N9 | (36) | | | | | | |
| 5 | N-61 | 5N6 | (35) | 5N61AB | (45) | 12 | U Msc I | 37 | 94 | 70 and 24 | HL60 |
| | (1, 2) | 3N9S | (46) | 3N9S | (36) | | | | | | |
| 6 | N-13 | 5N1S | (43) | 5BN4 | (29) | 10 | U Bsl I | 60 | 67 | 47 and 20 | oligo |
| | | 3N5S | (44) | 3N5S | (44) | | | | | | |
| 7 | K-61 | 5K25 | (22) | 5K22S | (18) | 8 | U Mse I | 37 | 132 | 107 and 25 | none |
| | (2, 3) | 3K23 | (27) | 3K23 | (27) | | | | | | |
| 7 | H-61 | 5HIN | (33) | 5HIP | (38) | 20 | U BstN I or | 37 | 98 | 75 and 23 | none |
| | | 3HIL | (34) | 3HIL | (34) | 12 | U Msc i | | | | |
| 7 | N-12 | 5N1 | (42) | 5BN2 | (28) | 10 | U Bsl I | 60 | 72 | 50 and 22 | Molt4 |
| | | 3N5 | (31) | 3N5 | (31) | | | | | | |
| 8 | N-12 | 5N1S | (43) | 5BN2 | (28) | 10 | U Bsl I | 60 | 74 | 50 and 24 | Molt4 |
| | | 3N5S | (44) | 3N5S | (44) | | | | | | |
| 9 | K-13 | 5KID | (10) | 5K37 | (30) | 10 | U Bsl I | 60 | 153 | 128 and 25 | HCT116 |
| | | 3KIE | (11) | 3KIU | (13) | | | | | | |
| 9 | H-12 | 5HIA | (39) | 5HIA | (39) | 20 | U Msp I | 37 | 117 | 72 and 45 | T24 |
| | | 3HIB | (41) | 3HIH | (40) | | | | | | |
| 9 | N-61 (3) | 5N6 | (35) | 5N14S | (23) | 15 | U Bfa I or | 37 | 97 | 72 and 25 | none |
| | | 3N9 | (36) | 3N9 | (36) | 4 | U Mae 1 | | | | |
| 10 | K-61 (1) | 5K25 | (22) | 5K40 | (32) | 10 | U Hae III | 37 | 134 | 111 and 23 | none |
| | | 3K23 | (27) | 3K23 | (27) | | | | | | |
| 11 | H intron | 5HID | (26) | 5HIJSC | (24) | 12 | U Mse I | 37 | 99 | 83 and 16 | T24 |
| | D | 3HIS | (25) | 3HIS | (25) | | | | | | |

*Numbers in parentheses in columns 3 and 4 indicate the SEQ ID NO of the identified primer.

The numerals in parentheses in column 2, for example K-61 specify the nucleotide bases screened for mutation in the 5′ to 3′ direction (codon 12 of K-ras, nucleotide bases 2 and 3). The absence of parentheses for K-12, K-13, N-12, N-13, and H-12 ras mutations indicates that the assay was capable of detecting a mutation at any of the first two nucleotide bases in the codon. In column 1, where the same assay number appears in multiple rows, a multiplexed assay for the specified target mutations was performed using the indicated primers in those rows. Where a single assay number appears in a single row only the specified target mutation was determined using the indicated primers in that row. Where the source of the target sequence in Table 2 is indicated as “none”, a cell line comprising the specified ras mutation was not available. In these cases, the size of the round 2 digestion products were determined based on restriction endonuclease digestion of the wild type DNA from cell-line K562.

Using the above-identified primers and methods, ras mutations were detected in cell lines or in oligonucleotides comprising ras mutation sequences. The size of digested and undigested products for each specific mutation are described in Table 2.

### EXAMPLE 6

REMS-PCR for N-12, N-13, and H-12 ras Mutations

For the detection of N-ras mutations at codons 12 and 13 and H-ras mutations at codon 12, each PCR admixture contained three sets of primers. The diagnostic primers induce a Bsl I restriction site in the wild type ras, but not in the indicated ras mutation at N-ras codon 12, N-ras codon 13, or H-ras codon 12. Thus, ras wild type DNA is selectively cleaved during PCR thermocycling, and mutant sequences of above indicated ras mutations are enriched. The PCR control primers were used to confirm that PCR amplifiable DNA was extracted, and the enzyme control primers confirmed that the restriction enzyme was functioning during thermocycling. Reaction admixtures contained 12 units/100 μL of recombinant Taq polymerase (developed at Ortho-Clinical Diagnostics), and a 2-fold molar excess (0.842 μL) of Taq inhibiting antibody TP4-9.2 the polymerase, HT50 buffer (100 mM sodium chloride, and 50 mM Tris-HCl, pH7.5), 0.2 μM of the indicated diagnostic primers pairs (see below), 0.05 μM of PCR control primer pairs 0.1 μM of enzyme control primer pairs (see below), 0.2 mM total dinucleoside triphosphates (dNTPs), 0.6 units/μL of Bsl I (New England BioLabs, Beverly Mass.), 1 mM dithiothreitol (DTT), 4 mM magnesium chloride, sample (typically 3 μL) and deionized water up to a final volume of 100 μL.

SEQ ID NO: 3 (5BK5) and SEQ ID NO: 4 (3K6) were used as a PCR control primer pair, and SEQ ID NO: 63 (5N12) and SEQ ID NO: 55 (3BN13) were used as the enzyme control primer pair. The following primer pairs were used: For the detection of H-ras mutation at codon 12: SEQ ID NO: 51 (5H12A) and SEQ ID NO:SEQ ID NO: 52 (3HB1); for the detection of H-ras mutation at codon 13: SEQ ID NO: 53 (5H13) and SEQ ID NO: 52 (3HB1); for the detection of N-ras mutation at codon 12: SEQ ID NO: 28 (5BN2) and SEQ ID NO: 31 (3N5); and for the detection of N-ras at codon 13: SEQ ID NO: 29 (5N4) and SEQ ID NO:SEQ ID NO: 31 (3N5).

Thermocycling was performed on a Gene-Amp 9600 (Perkin-Elmer, Norwalk, Conn.) with the following parameters for Round 1: 1 cycle of 94° C. for 3 mins, followed by 32 cycles of alternate incubations at 94° C. for 10 sec, and 60° C. for 30 sec. For the assay of mutations in H-ras intron D, 38 cycles of PCR amplification were used instead of 32 cycles.

The following primers were used in REMS-PCR with Bsl I as restriction enzyme for determining mutations at N-ras codons 12 and 13 and mutations at H-ras codon 12.

| | |
|---|---|
| AATATAAGCT GGTGGTGCCG GGCG | SEQ ID NO: 48 |
| ATAAGCTGGT GGTGCCCGCC CT | SEQ ID NO: 49 |
| TGAATATAAA CTTGTGGTAC CTGGAGCT | SEQ ID NO: 50 |
| AATATAAGCT GGTGGTGCCG GGCGC | SEQ ID NO: 51 |
| AATGGTTCTG GATCAGCTGG ATG | SEQ ID NO: 52 |
| ATAAGCTGGT GGTGGTGCCC GCCG | SEQ ID NO: 53 |
| TATAGATGGT GAAACCTGTT TGTTGG | SEQ ID NO: 54 |
| CTATTATTGA TGGCAACCAC ACAG | SEQ ID NO: 55 |

Using the above-identified primers and methods, ras mutations were detected in cell lines or in oligonucleotides comprising ras mutation sequences.

### EXAMPLE 7

Detection of H-12, N-12 and N-13 ras Mutations Based on a Molecular Beacon Assay

The following probes were used for determining mutations in H-12, N-12 and N-13. The probe for H-12 can also be used for detecting H-13 mutations. The target specific probes were prepared in the form of molecular beacons comprising the fluor FAM at the 5′ terminus and the quencher DABCYL at the 3′ terminus. Primers from example 5 were used in REMS-PCR.

SEQ ID NO: 56

GCGAGCGTGG TGTTGGGAAA AGCGCAGCTC GC

SEQ ID NO: 57

GCGAGCCGTC GGTGTGGGCA GAGTGCGCTG CTCGC

SEQ ID NO: 58

GCGAGCGAAA CCTCAGCCAA GACCAGACAG GCTCGC

SEQ ID NO: 59

GCGAGCGACA TAACAGTTAT GATTTTGCAG AAAACAGATC
GCTCGC

SEQ ID NO: 60

ATATAAACTT GTGGTACCTG GAGCT

SEQ ID NO: 61

TATAGATGGT GAAACCTGTT TG

SEQ ID NO: 62

CTTGCTATTA TTGATGGCAA CCACACAGA

SEQ ID NO: 63

TATAGATGGT GAAACCTGTT TG

SEQ ID NO: 64

ATAAGCTGGT GGTGCCGGGC G

SEQ ID NO: 65

ATGGTTCTGG ATCAGCTGG

SEQ ID NO: 66

AATATAAGCT GGTGGTGCCG GGCG

SEQ ID NO: 67

AATGGTTCTG GATCAGCTGG ATGGTC

Reaction admixtures contained 12 units/100 μL of recombinant Taq polymerase (developed at Ortho-Clinical Diagnostics), and a 4-fold molar excess (0.842 μL) of Taq inhibiting antibody TP4-9.2 the polymerase, HT50 buffer (100 mM sodium chloride, and 50 mM Tris-HCl , pH7.50.2 mM total dinucleoside triphosphates (dNTPs), 0.3 units/μL of Bsl I (New England BioLabs, Beverly Mass.), 1 mM dithiothreitol (DTT), 4 mM magnesium chloride, sample composed of the indicated dilution of mutant and wild type DNA (typically 3 μL) and deionized water up to a final volume of 100 μL.

The following primers and molecular beacons were added to individual reaction admixtures for the detection of N-12 ras mutations based on REMS-PCR and molecular beacons. PCR control primers 5BK5 (SEQ ID NO:SEQ ID NO: 3) and 3K6 (SEQ ID NO: 4) were added at 0.1 μM, 0.2 μM enzyme control primers 5N12A (SEQ ID NO: 54) and 3N13A (SEQ ID NO: 49), and 0.3 μM diagnostic primers 5BN2 (SEQ ID NO:28) and 3N5S (SEQ ID NO: 44). The PCR control molecular beacon was BPIG (SEQ ID NO: 15), the enzyme control molecular beacon was BE1 (SEQ ID NO:16) and the diagnostic molecular beacon was BND12 (SEQ ID NO: 56) All molecular beacons were added at 0.2 uM each and were labeled with a FAM dye. PCR thermal cycling parameters on the ABI Prism 7700 Sequence Detector were as described in example 3 except that 45 PCR cycles were used.

The following primers and molecular beacons were added to individual reaction admixtures for the detection of H-12 ras mutations based on REMS-PCR and molecular beacons. PCR control primers 5BK5 (SEQ ID NO:SEQ ID NO: 3) and 3K6 (SEQ ID NO: 4) were added at 0.0.05 μM, 0.1 μM enzyme control primers 5N12A (SEQ ID NO: 54) and 3N13A (SEQ ID NO: 49), and 0.2 μM diagnostic primers 5H12B (SEQ ID NO: 66) and 3HB2 (SEQ ID NO: 67). The PCR control molecular beacon was BP1 (SEQ ID NO: 59), the enzyme control molecular beacon was BE1 (SEQ ID NO: 16) and the diagnostic molecular beacon was BHD12 (SEQ ID NO: 57) All molecular beacons were added at 0.2 uM each and were labeled with a FAM dye. PCR thermal cycling on the ABI Prism 7700 Sequence Detector were as described in example 3 except that a total of 45 thermal cycles were used.

The following primers and molecular beacons were added to individual reaction admixtures for the detection of N ras mutations at codon 13 based on REMS-PCR and molecular beacons. PCR control primers 5BK5 (SEQ ID NO: 3) and 3K6 (SEQ ID NO: 4) were added at 0.1 μM, 0.1 μM enzyme control primers 5N12A (SEQ ID NO: 54) and 3N13 (SEQ ID NO: 55), and 0.3 μM diagnostic primers 5N4 (SEQ ID NO: 29) and 3N5S (SEQ ID NO: 44). The PCR control molecular beacon was BPI (SEQ ID NO: 59), the enzyme control molecular beacon was BEL (SEQ ID NO: 58) and the diagnostic molecular beacon was BND12 (SEQ ID NO: 56) All molecular beacons were added at 0.2 uM each and were labeled with a FAM dye. PCR thermal cycling parameters on the ABI Prism 7700 Sequence Detector were as described in example 3 except that 45 thermal cyles were used.

Results of these studies are shown below in Table 3. All PCR controls exhibited a Ct value between 28.31 and 34.43 indicating that samples contained PCR amplifiable DNA. All diagnostic samples for H-ras mutations at codon 12 and N-ras mutations at codons 12 and 13 exhibited Ct values values below 40 except for the most dilute 1:1000 mutant to wild type sample for N-ras mutation at codon 12, indicating that mutant sequences were detectable. Enzyme control samples exhibited a Ct of >40 or >45, except for a control sample in which restriction enzyme was deleted. This sample had a Ct value of 29.1 indicating that the enzyme control functioned in the absence of restriction enzyme. These results indicate that the ras mutations can be detected at dilutions of 1:100 mutant to wild type or greater based on the above-identified primers, probes and methods.

TABLE 3
Homogeneous PCR Detection of H-ras Mutations at
Codon 12 and N-ras Mutations at Codons 12 and 13

| | | | Enzyme | |
|---|---|---|---|---|
| | Mutant:Wild | Diagnostic | Control | PCR Control |
| Mutation | Type | c_{T} | C_{T} | C_{T} |
| H-ras | 1:1000 | 38.15 | >40.00 | 28.31 |
| Codon 12 | 1:100 | 35.44 | >40.00 | 28.99 |
| | 1:10 | 30.26 | >40.00 | 29.37 |
| | 0 | >40.00 | >40.00 | 30.98 |
| N-ras | 1:10000 | 29.47 | >40.00 | 34.43 |
| Codon 13 | 1:1000 | 26.21 | >40.00 | 34.25 |
| | 1:100 | 21.59 | >40.00 | 34.71 |
| | 0 | >40.00 | >40.00 | 33.93 |
| N-ras | 1:1000 | >45.00 | >45.00 | 29.59 |
| Codon 12 | 1:100 | 38.43 | >45.00 | 29.04 |
| | 1:10 | 36.19 | >45.00 | 28.91 |
| | 0 | >44.46 | >45.00 | 31.87 |
| Control | ND | ND | 29.01 | ND |
| (no | | | | |
| restriction | | | | |
| endonuclease) | | | | |

The invention has been described in detail with respect to particular preferred embodiments. It will be understood that variations and modifications can be effected without departing from the scope and spirit of the invention. The entire contents of all patents, patent applications, and non-patent disclosures and their citations are expressly incorporated herein by reference.

We claim:

1. One or more oligonucleotides comprising a sequence selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67.

2. One or more oligonucleotides comprising a sequence selected from SEQ ID NO:5, SEQ ID NO:6, SEQ ID NO:7, SEQ ID NO:8, or SEQ ID NO:9.

3. One or more oligonucleotides comprising a sequence selected from SEQ ID NO:10, SEQ ID NO:12, or SEQ ID NO:13.

4. One or more oligonucleotides comprising a sequence selected from SEQ ID NO:14, SEQ ID NO:15, SEQ ID NO:16, or SEQ ID NO:17.

5. One or more oligonucleotides comprising a sequence selected from SEQ ID NO:18, SEQ ID NO:19, SEQ ID NO:20, or SEQ ID NO:21.

6. One or more oligonucleotides comprising a sequence selected from SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, or SEQ ID NO:47.

7. One or more oligonucleotides comprising a sequence selected from SEQ ID NO:48, SEQ ID NO:49, SEQ ID NO:50, SEQ ID NO:51, SEQ ID NO:52, SEQ ID NO:53, SEQ ID NO:54, or SEQ ID NO:55.

8. One or more oligonucleotides comprising a sequence selected from SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67.

9. The oligonucleotides of claim 4 further comprising one or more fluorescence moieties and one or more fluorescence quenching moieties.

10. The oligonucleotides of claim 9 wherein
(i) one or more fluorescence moieties are linked to one or more nucleotides adjacent to the 3′ terminal nucleotide or linked to the 3′ terminal nucleotide or both and one or more fluorescence quenching moieties are linked to one or more nucleotides adjacent to the 5′ terminal nucleotide or linked to the 5′ terminal nucleotide or both, and wherein one or more nucleolides comprising the 3′ terminus are complementary to one or more nucleotides comprising the 5′ terminus, or
(ii) one or more fluorescence moieties are linked co one or more nucleotides adjacent to the 5′ terminal nucleotide or linked to the 5′ terminal nucleotide or both, and one or more fluorescence quenching moieties are linked to one or more nucleotides adjacent to the 3′ terminal nucleotide or linked to the 3′ terminal nucleotide or both, and wherein one or more nucleotides comprising the 3′ terminus are complementary to one or more nucleotides comprising the 5′ terminus.

11. The oligonucleotides of claim 10 wherein the fluorescence moiety is carboxyfluorescein, carboxy-4′,5′-dichloro-2′,7′-dimethoxyfluorescein, tetrachlorofluorescein, dimethoxyfluorescein, or carboxyrhodamine and the quenching moiety is (4-(4′dimethylaminophenylazo)benzoic acid) or 4(dimethylamine)azobenzene sulfonic acid.

12. One or more oligonucleotides comprising a sequence selected from SEQ ID NO:19, SEQ ID NO:20, or SEQ ID NO:21, said one or more oligonucleotides comprising one or more fluorescence moieties and one or more fluorescence quenching moieties.

13. The oligonucleotides of claim 12 wherein
(i) one or more fluorescence moieties are linked to one or more nucleotides adjacent to the 3′ terminal nucleotide or linked to the 3′ terminal nucleotide or both and one or more fluorescence quenching moieties are linked to one or more nucleotides adjacent to the 5′ terminal nucleotide or linked to the 5′ terminal nucleotide or both, and wherein one or more nucleotides comprising the 3′ terminus are complementary to one or more nucleotides comprising the 5′ terminus, or
(ii) one or more fluorescence moieties are linked to one or more nucleotides adjacent to the 5′ terminal nucleotide or linked to the 5′ terminal nucleotide or both and one or more fluorescence quenching moieties are linked to one or more nucleotides adjacent to the 3′ terminal nucleotide or linked to the 3′ terminal nucleotide or both, and wherein one or more nucleotides comprising the 3′ terminus are complementary to one or more nucleotides comprising the 5′ terminus.

14. The oligonucleotides of claim 13 wherein the fluorescence moiety is carboxyfluorescein, carboxy-4′,5′-dichloro-2′,7′-dimethoxyfluorescein, tetrachlorofluorescein, dimethoxyfluorescein, or carboxyrhodamine and the quenching moiety is (4-(4′dimethylaminophenylazo)benzoic acid) or 4(dimethylamine)azobenzene sulfonic acid.

15. The oligonucleotides of claim 8 further comprising one or more fluorescence moieties and one or more fluorescence quenching moieties.

16. The oligonucleotides of claim 15
(i) one or more fluorescence moieties are linked to one or more nucleotides adjacent to the 3′ terminal nucleotide or linked to the 3′ terminal nucleotide or both and one or more fluorescence quenching moieties are linked to one or more nucleotides adjacent to the 5′ terminal nucleotide or linked to the 5′ terminal nucleotide or both, and wherein one or more nucleotides comprising the 3′ terminus are complementary to one or more nucleotides comprising the 5′ terminus, or
(ii) one or more fluorescence moieties are linked to one or more nucleotides adjacent to the 5′ terminal nucleotide or linked to the 5′ terminal nucleotide or both and one or more fluorescence quenching moieties are linked to one or more nucleotides adjacent to the 3′ terminal nucleotide or linked to the 3′ terminal nucleotide or both, and wherein one or more nucleotides comprising the 3′ terminus are complementary to one or more nucleotides comprising the 5′ terminus.

17. The oligonucleotides of claim 16 wherein the fluorescence moiety is carboxyfluorescein, carboxy-4′,5′-dichloro-2′,7′-dimethoxyfluorescein, tetrachlorofluorescein, dimethoxyfluorescein, or carboxyrhodamine and the quenching moiety is (4-(4′dimethylaminophenylazo)benzoic acid) or 4(dimethylamine)azobenzene sulfonic acid.

18. A method for amplifying DNA comprising a mutant ras sequence in a sample comprising the steps of:
(B) forming an admixture comprising
(i) the sample,
(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,
(iii) at least four different nucleoside triphosphates, one or more thermostable polymerases, and at least one thermostable restriction endonuclease that is capable of directly cleaving wild type K-, H-, or N-ras sequence or cleaving a primer induced cleavage site, or both; and
(B) subjecting the admixture to one or more cycles of heating and cooling.

19. A method for determining one or more ras mutations in a DNA sample comprising the steps of:
(A) forming an admixture comprising
(i) the sample,
(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,
(iii) at least four different nucleoside triphosphates, one or more thermostable polymerases, and at least one thermostable restriction endonuclease that is capable of directly cleaving wild type K-, H-, or N-ras sequence or cleaving a primer induced cleavage site, or both; and
(B) subjecting the admixture to one or more cycles of heating and cooling;
(C) separating the DNA by electrophoresis; and
(D) detecting the DNA comprising-a mutant ras sequence separated by electrophoresis in step (C).

20. A method for determining one or more ras mutations in a DNA sample comprising the steps of:
(A) forming an admixture comprising
(i) the sample,
(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,
(ii) at least four different nucleoside triphosphates, one or more thermostable polymerases, and at least one thermostable restriction endonuclease that is capable of directly cleaving wild type K-, H-, or N-ras sequence or cleaving a primer induced cleavage site, or both; and
(B) subjecting the admixture to one or more cycles of heating and cooling;
(C) combining the admixture comprising amplified DNA with one or more immobilized oligonucleotides or one or more oligonucleotides capable of being immobilized, said one or more oligonucleotides being capable of hybridizing to DNA comprising a mutant ras sequence thereby capturing DNA comprising a mutant ras sequence; and
(D) detecting the captured DNA comprising a mutant ras sequence.

21. The method of claim 20 wherein the sequence of one or more immobilized oligonucleotides or one or more oligonucleotides capable of being immobilized is selected from SEQ ID NO:7, SEQ ID NO:8, or SEQ ID NO:9.

22. A method for determining one or more ras mutations in a DNA sample comprising the steps of:
(A) forming an admixture comprising
(i) the sample,
(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,
(iii) at least four different nucleoside triphosphates, one or more thermostable polymerases, and at least one thermostable restriction endonuclease that is capable of directly cleaving wild type K-, H-, or N-ras sequence or cleaving a primer induced cleavage site, or both, and
(iv) one or more oligonucleotides comprising one or more fluorescence moieties and one or more fluorescence quenching moieties, said one or more oligonucleotides being capable of hybridizing to DNA comprising a mutant ras sequence and capable of producing detectable fluorescence when hybridized thereto;
(B) subjecting the admixture to one or more cycles of heating and cooling;
(C) detecting the fluorescence.

23. The method of claim 22 wherein
(i) the one or more fluorescence moieties are linked to one or more nucleotides adjacent to the 3′ terminal nucleotide or linked to the 3′ terminal nucleotide or both and the one or more fluorescence quenching moieties are linked to one or more nucleotides adjacent to the 5′ terminal nucleotide or the one or more fluorescence quenching moieties are linked to the 5′ terminal nucleotide or both, and wherein one or more nucleotides comprising the 3′ terminus are complementary to one or more nucleotides comprising the 5′ terminus, or
(ii) the one or more fluorescence moieties are linked to one or more nucleotides adjacent to the 5′ terminal nucleotide or linked to the 5′ terminal nucleotide or both, and the one or more fluorescence quenching moieties are linked to one or more nucleotides adjacent to the 3′ terminal nucleotide or linked to the 3′ terminal nucleotide or both, and wherein one or more nucleotides comprising the 3′ terminus are complementary to one or more nucleotides comprising the 5′ terminus.

24. The method of claim 23 wherein the fluorescence moiety is carboxyfluorescein, carboxy-4′,5′-dichloro-2′,7′-dimethoxyfluorescein, tetrachlorofluorescein, dimethoxyfluorescein, or carboxyrhodamine and the quenching moiety is (4-(4′dimethylaminophenylazo)benzoic acid) or 4(dimethylamine)azobenzene sulfonic acid.

25. A method for amplifying DNA comprising a mutant ras sequence in a sample comprising the steps of:
(A) forming an admixture comprising
(i) the sample,
(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,
(iii) at least four different nucleoside triphosphates and one or more thermostable polymerases;
(B) subjecting the admixture to one or more cycles of heating and cooling;
(C) combining the admixture with one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NC:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, provided at least one of the primers is different from the primers in step (A);
(D) subjecting the admixture to one or more cycles of heating and cooling; and
(E) combining the admixture with at least one restriction endonuclease that is capable of directly cleaving wild type K-, H-, or N-ras sequence or cleaving a primer induced cleavage site, or both.

26. A method for determining one or more ras mutations in a DNA sample comprising the steps of:
(A) forming an admixture comprising
(i) the sample,
(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,
(iii) at least four different nucleoside triphosphates and one or more thermostable polymerases;
(B) subjecting the admixture to one or more cycles of heating and cooling;
(C) combining the admixture with one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, provided at least one of the orimers is different from the primers in step (A);
(D) subjecting the admixture to one or more cycles of heating and cooling; and
(E) combining the admixture with at least one restriction endonuclease that is capable of directly cleaving wild type K-, H-, or N-ras sequence or cleaving a primer induced cleavage site, or both;
(F) separating the DNA by electrophoresis; and
(G) detecting the DNA comprising a mutant ras sequence separated by electrophoresis in step (F).

27. A method for determining one or more ras mutations in a DNA sample comprising the steps of:
(A) forming an admixture comprising
(i) the sample,
(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,
(iii) at least four different nucleoside triphosphates and one or more thermostable polymerases;
(B) subjecting the admixture to one or more cycles of heating and cooling;
(C) combining the admixture with one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, provided at least one of the primers is different from the primers in step (A);
(D) subjecting the admixture to one or more cycles of heating and cooling; and
(E) combining the admixture with at least one restriction endonuclease that is capable of directly cleaving wild type K-, H-, or N-ras sequence or cleaving a primer induced cleavage site, or both;
(F) combining the admixture comprising amplified DNA with one or more immobilized oligonucleotides or oligonucleotides capable of being immobilized, said oligonucleotides being capable of hybridizing to DNA comprising a mutant ras sequence thereby capturing DNA comprising a mutant ras sequence; and
(G) detecting the captured DNA comprising a mutant ras sequence.

28. A method for determining one or more ras mutations in a DNA sample comprising the steps of:
(A) forming an admixture comprising
(i) the sample,
(ii) one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:30, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67,
(iii) at least four different nucleoside triphosphates, and one or more thermostable polymerases;
(iv) one or more oligonucleotides comprising one or more fluorescence moieties and one or more fluorescence quenching moieties, said one or more oligonucleotides being capable of hybridizing to DNA comprising a mutant ras sequence and capable of producing detectable fluorescence when hybridized thereto;
(B) subjecting the admixture to one or more cycles of heating and cooling;
(C) combining the admixture produced in step (B) with one or more primer pairs selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, SEQ ID NO:55, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, provided at least one of the primers is different from the primers in step (A);
(D) subjecting the admixture to one or more cycles of heating and cooling;
(E) combining the admixture with at least one restriction endonuclease that is capable of directly cleaving wild type K-, H-, or N-ras sequence or cleaving a primer induced cleavage site, or both; and
(F) detecting the fluorescence.

29. The method of claim 28 wherein
(i) the one or more fluorescence moieties are linked to one or more nucleotides adjacent to the 3′ terminal nucleotide or linked to the 3′ terminal nucleotide or both and the one or more fluorescence quenching moieties are linked to one or more nucleotides adjacent to the 5′ terminal nucleotide or the one or more fluorescence quenching moieties are linked to the 5′ terminal nucleotide or both, and wherein one or more nucleotides comprising the 3′ terminus are complementary to one or more nucleotides comprising the 5′ terminus, or
(ii) the one or more fluorescence moieties are linked to one or more nucleotides adjacent to the 5′ terminal nucleotide or linked to the 5′ terminal nucleotide or both and the one or more fluorescence quenching moieties are linked to one or more nucleotides adjacent to the 3′ terminal nucleotide or linked to the 3′ terminal nucleotide or both, and wherein one or more nucleotides comprising the 3′ terminus are complementary to one or more nucleotides comprising the 5′ terminus.

30. The method of claim 28 wherein the fluorescence moiety is carboxyfluorescein, carboxy-4′,5′-dichloro-2′,7′-dimethoxyfluorescein, tetrachlorofluorescein, dimethoxyfluorescein, or carboxyrhodamine and the quenching moiety is (4-(4′dimethylaminophenylazo)benzoic acid) or 4(dimethylamine)azobenzene sulfonic acid.

31. A kit comprising in one or more containers:
(i) one or more oligonucleotides comprising a sequence selected from SEQ ID NO:5, SEQ ID NO:6, SEQ ID NO:7, SEQ ID NO:8, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, SEQ ID NO:33, SEQ ID NO:34, SEQ ID NO:35, SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, SEQ ID NO:49, SEQ ID NO:50, SEQ ID NO:51, SEQ ID NO:52, SEQ ID NO:53, SEQ ID NO:54, or SEQ ID NO:55;
(ii) one or more oligonucleotides selected from SEQ ID NO:19, SEQ ID NO:20, SEQ ID NO:21, SEQ ID NO:56, SEQ ID NO:57, SEQ ID NO:58, SEQ ID NO:59, SEQ ID NO:60, SEQ ID NO:61, SEQ ID NO:62, SEQ ID NO:63, SEQ ID NO:64, SEQ ID NO:65, SEQ ID NO:66, or SEQ ID NO:67, the oligonucleotide or oligonucleotides comprising one or more fluorescence moieties and one or more fluorescence quenching moieties.

32. The kit of claim 31 wherein
(i) the one or more fluorescence moieties are linked to one or more nucleotides adjacent to the 3′ terminal nucleotide or linked to the 3′ terminal nucleotide or both and the one or more fluorescence quenching moieties are linked to one or more nucleotides adjacent to the 5′ terminal nucleotide or the one or more fluorescence quenching moieties are linked to the 5′ terminal nucleotide or both, and wherein one or more nucleotides comprising the 3′ terminus are complementary to one or more nucleotides comprising the 5′ terminus, or
(ii) the one or more fluorescence moieties are linked to one or more nucleotides adjacent to the 5′ terminal nucleotide or linked to the 5′ terminal nucleotide or both and the one or more fluorescence quenching moieties are linked to one or more nucleotides adjacent to the 3′ terminal nucleotide or linked to the 3′ terminal nucleotide or both, and wherein one or more nucleotides comprising the 3′ terminus are complementary to one or more nucleotides comprising the 5′ terminus.

33. The kit of claim 32 wherein the fluorescence moiety is carboxyfluorescein, carboxy-4′,5′-dichloro-2′,7′-dimethoxyfluorescein, tetrachlorofluorescein, dimethoxyfluorescein, or carboxyrhodamine and the quenching moiety is (4-(4′dimethylaminophenylazo)benzoic acid) or 4(dimethylamine)azobenzene sulfonic acid.

34. The kit of claim 33 further comprising in one or more containers
(i) one or more nucleoside triphosphates;
(ii) one or more restriction endonucleases capable of directly cleaving wild type K-, H-, or N-ras sequence or a primer induced cleavage site, or both; and
(iii) one or more thermostable polymerases.

35. A method for amplifying and determining one or more target mutant sequences in a DNA sample comprising the steps of:
(A) forming an admixture comprising
(i) the sample,
(ii) one or more primer pairs specific for one or more of the mutant target sequences,
(iii) at least four different nucleoside triphosphates,
(iv) one or more thermostable polymerases,
(v) at least one thermostable restriction endonuclease that is capable of directly cleaving wild type DNA sequence of the mutant target or mutant targets or cleaving a primer induced cleavage site, or both,
(vi) one or more oligonucleotides comprising one or more fluorescence moieties and one or more fluorescence quenching moieties, said one or more oligonucleotides being capable of hybridizing to DNA comprising the target mutant sequence or target mutant sequences and capable of producing detectable fluorescence when hybridized thereto;
(B) subjecting the admixture to one or more cycles of heating and cooling;
(C) detecting the fluorescence.

36. The method of claim 35 wherein
(i) the one or more fluorescence moieties are linked to one or more nucleotides adjacent to the 3′ terminal nucleotide or linked to the 3′ terminal nucleotide or both and the one or more fluorescence quenching moieties are linked to one or more nucleotides adjacent to the 5′ terminal nucleotide or the one or more fluorescence quenching moieties are linked to the 5′ terminal nucleotide or both, and wherein one or more nucleotides comprising the 3′ terminus are complementary to one or more nucleotides comprising the 5′ terminus, or
(ii) the one or more fluorescence moieties are linked to one or more nucleotides adjacent to the 5′ terminal nucleotide or linked to the 5′ terminal nucleotide or both and the one or more fluorescence quenching moieties are linked to one or more nucleotides adjacent to the 3′ terminal nucleotide or linked to the 3′ terminal nucleotide or both, and wherein one or more nucleotides comprising the 3′ terminus are complementary to one or more nucleotides comprising the 5′ terminus.

37. The method of claim 36 wherein the fluorescence moiety is carboxyfluorescein, carboxy-4′,5′-dichloro-2′,7′-dimethoxyfluorescein, tetrachlorofluorescein, dimethoxyfluorescein, or carboxyrhodamine and the quenching moiety is (4-(4′dimethylaminophenylazo)benzoic acid) or 4(dimethylamine)azobenzene sulfonic acid.
